(19) United States
(12) Patent Application Publication
(10) Pub. No.: US 2026/0000763 A1
(43) Pub. Date: Jan. 1, 2026

(54) Methods of Manufacturing Therapeutic Cells

(71) Applicant: The Board of Trustees of the Leland Stanford Junior University, Stanford, CA (US)

(72) Inventors: Crystal Mackall, Redwood City, CA (US); Steven Feldman, Redwood City, CA (US); Dorota Klysz, Redwood City, CA (US)

(21) Appl. No.: 18/881,674

(22) PCT Filed: Jul. 7, 2023
(86) PCT No.: PCT/US2023/027171

§ 371 Date: Jan. 6, 2025

Related U.S. Application Data
(60) Provisional application No. 63/358,996, filed on Jul. 7, 2022.
(60) Provisional application No. 63/440,576, filed on Jan. 23, 2023.

(51) Int. Cl. A61K 40/31; A61K 40/11; A61K 40/42; A61P 35/00; C07K 14/725; C07K 16/32; C12N 5/0783

(52) U.S. Cl. CPC A61K 40/31; A61K 40/11; A61K 40/4202; A61P 35/00; C07K 14/7051; C07K 16/32; C12N 5/0636; C12N 2501/999

(57) ABSTRACT

Provided are methods of manufacturing cells. The methods comprise expanding cells in the presence of inosine, and harvesting the cells after expansion. In certain embodiments, the cells are genetically modified. For example, the cells may be genetically modified to express an engineered receptor, non-limiting examples of which are chimeric antigen receptors (CARs), engineered T cell receptors (TCRs), and the like. According to some embodiments, the cells are immune cells, e.g., T cells or natural killer cells. Also provided are populations of cells manufactured according to the methods of the present disclosure. Also provided are methods comprising administering an effective amount of a population of cells manufactured according to the methods of the present disclosure to a subject in need thereof. In certain embodiments, the subject has cancer, and the cells express an engineered receptor that binds to a tumor antigen on the surface of cells of the cancer.

### CROSS-REFERENCE TO RELATED APPLICATIONS

This application claims the benefit of U.S. Provisional Patent Application No. 63/440,576, filed Jan. 23, 2023, and U.S. Provisional Patent Application No. 63/358,996, filed Jul. 7, 2022, which applications are incorporated herein by reference in their entireties.

### STATEMENT OF GOVERNMENT SUPPORT

This invention was made with Government support under contract CA124435 awarded by the National Institutes of Health. The Government has certain rights in the invention.

### SUMMARY

Provided are methods of manufacturing cells. The methods comprise expanding cells in the presence of inosine, and harvesting the cells after expansion. In certain embodiments, the cells are genetically modified. For example, the cells may be genetically modified to express an engineered receptor, non-limiting examples of which are chimeric antigen receptors (CARs), engineered T cell receptors (TCRs), and the like. According to some embodiments, the cells are immune cells, e.g., T cells or natural killer cells. Also provided are populations of cells manufactured according to the methods of the present disclosure. Also provided are methods comprising administering an effective amount of a population of cells manufactured according to the methods of the present disclosure to a subject in need thereof. In certain embodiments, the subject has cancer, and the cells express an engineered receptor that binds to a tumor antigen on the surface of cells of the cancer.

### BRIEF DESCRIPTION OF THE FIGURES

FIG. 1A-1G: Data demonstrating that in vitro cell expansion in the presence of inosine increases the effector function of non-exhausted CAR T cells in vitro. In this example, CD19.28 CAR T cells were cultured in regular complete RPMI media until day 4, and then the cells were split between 11 mM glucose-(CTR) or 11 mM inosine-(INO) containing media (unless stated otherwise). A: Annexin 5 staining was performed at day 17 post-activation. Representative histograms of n=3 donors are shown. B: Surface CD19.28 CAR expression at day 14 post-activation. C: MFI of CAR molecules on CD19.28z CAR T cell surface at day 14 post activation. D: Ratio of CD4+vs CD8+CD19.28 CAR T cells at day 14 post-activation. E: CD19.28z CAR T cells were co-cultured with cells of the Nalm6 tumor line (1:8 E: T) for 75 hrs in an IncuCyte assay. Tumor GFP fluorescence intensity was normalized to the first time point (triplicate wells). Representative donor of n=3 is shown. F: Control media was replaced by INO+ media at the indicated day post activation. At day 14, cells were stimulated with Nalm6 tumor line, and IL-2 and IFNγ secretion was assessed by ELISA. G: IFNγ secretion by CAR T cells stimulated with Nalm6 tumor line expressing medium or low levels of CD19 antigen, respectively, at day 14 post-activation. For A and B, P values were determined by paired two-tailed t-tests. For E, P values were determined by two-way ANOVA with Dunnett's multiple comparisons test. For F and G, P values were determined by unpaired two-tailed t-tests.

FIG. 2A-2C: Data demonstrating that in vitro cell expansion in the presence of inosine increases the effector function of exhausted CAR T cells in vitro. A: CD4+vs CD8+ ratio at day 14 post-activation. N-7 donors. P values were determined by paired two-tailed t-tests. B: Co-culture with Nalm6-GD2 (1:8 E: T), 143b (1:1 E: T) and CHL255 (1:10 E: T) tumor cell lines in an IncuCyte assay to assess CAR T cell cytotoxicity. Tumor GFP fluorescence intensity was normalized to the first time point (triplicate wells). P values were determined by two-way ANOVA with Dunnett's multiple comparisons test. C: IL-2 and IFNγ secretion at day 14 post-activation. P values were determined by unpaired two-tailed t-tests.

FIG. 3A-3D: Data demonstrating that increased inosine metabolism results in higher frequency of memory/naïve and decreased frequency of terminally differentiated CAR T cells. In this example, CAR T cells were cultured in control media until day 4 post activation, followed by continued expansion in regular or inosine-containing media. Phenotype of the cells was assessed by flow cytometry at day 21 post activation. A: Frequency of CD62L+ and CCR7+HA CAR+ T cells in 8 different donors. Representative histograms are shown. B: Relative frequency of central memory (CM), naïve, terminally differentiated (TEMRA) and effector memory (EM) HA CAR T cells. N=8 donors. C: Frequency of CD62L+ and CCR7+CD19.28 CAR+ T cells in 8 different donors. Representative histograms are shown. D: Relative frequency of central memory (CM), naïve, terminally differentiated (TEMRA) and effector memory (EM) CD19.28 CAR T cells. N=8 donors. For A-D, P values determined by paired two-tailed t-tests.

FIG. 4A-4D: Data demonstrating that inosine changes the metabolic phenotype of CAR T cells. A and B: Seahorse analysis of CAR T cells at day 11 post-activation. Representative graphs of n=1-2 donors are shown. P values were determined by unpaired two-tailed t-tests. C: Flow analysis of ATP5 expression by CD8+ CAR T cells at day 17 post-activation. Representative histograms of two donors are shown. D: ATP levels in CAR T cells at day 14 post activation. Results from two independent experiments are shown (n=2).

FIG. 5A-5E: Data demonstrating that CAR T cells manufactured in inosine-containing media exhibit increased anti-tumor function in vivo. A: NSG mice were injected IV with 1×10⁶ Nalm6 leukemia cells at day 0, and then 0.2×10⁶ CD19.28 CAR T cells, generated in regular or inosine-supplemented media were given IV on day 3. Tumor progression was monitored using bioluminescent imaging. Scales are normalized for all time points. Data are mean+s.e.m. of n=5 mice per group. P values were determined by two-way ANOVA with Dunnett's multiple comparisons test. B: NSG mice were injected IV with 1×10⁶ Nalm6-GD2+ leukemia cells at day 0, and then 2×10⁶ HA CAR T cells, generated in regular or inosine-containing media were given IV on day 3. Tumor progression was monitored using bioluminescent imaging. Scales are normalized for all time points. Data are mean+s.e.m. of n=5 mice per group. P values were determined by two-way ANOVA with Dunnett's multiple comparisons test. C: Overall survival. N=5 mice per group. Survival curves were compared using the log-rank Mantel-Cox test. D: NSG mice were injected intramuscular with 1×10⁶ 143b tumor cells. 4 days later the mice were injected with 1×10⁷ Her2, Her2 grown in INO+ media or mock T cells IV. Tumor growth was monitored by caliper measurements. P values were determined by two-way ANOVA with Dunnett's multiple comparisons test. E: INO+ Her2.bb CAR T cells induced long-term tumor-free survival. N=3-5 mice per group. Survival curves were compared using the log-rank Mantel-Cox test.

FIG. 6A-6G: Data demonstrating that CAR T cells manufactured in inosine-containing media at a large, clinical-scale exhibit increased anti-tumor function. Enriched CD4/CD8+ T cells from a healthy donor were cultured in regular or inosine-containing media in the G-Rex system. Cells were analyzed at days 7 post-activation. A: Scheme of the large scale experiment. B: Expansion of GD2 CAR T cells manufactured in control vs inosine-containing media. N=1 out of two donors is shown. C: Number of CAR+ T cells at the day 7 post activation. D: Viability of T cells. E: Percentage of CAR+ and CD4 vs CD8 T cells. Data from one representative donor is shown. N=2. F: Relative frequency of terminally differentiated effector (EMRA; CD45RA+ CCR7-), naïve (CD45RA+ CCR7+), central memory (CM; CD45RA-CCR7+), and effector memory (EM; CD45RA-CCR7-) cells in CD8+ control or inosine-grown GD2 CAR T cells. G: IL-2 and IFNγ secretion by CAR T cells stimulated with 143b, mg63.3 or Nalm6-GD2 tumor lines. Error bars represent mean+SD of triplicate wells from one representative donor (n=2 donors). P values were determined by unpaired two-tailed t-tests.

FIG. 7A: Schematic of experimental design.

FIG. 7B: (Left) Cell expansion of HA-CAR T cells grown in culture media containing indicated concentration of glucose and/or inosine from day 4 to day 10 post-activation. Data are mean±s.e.m. of n=2-4 donors. P values determined by paired two-tailed t-tests. (Right) Expansion of HA-CAR T cells grown in RPMI containing 11 mM of glucose and increasing concentrations of inosine for 6 days. Scatter plot showing correlation between ratio of expansion (y-axis) and inosine concentration (x-axis). Pearson correlation, r, with P values determined by paired two-tailed t-tests. N=2-4 donors from independent experiments.

FIG. 7C: (Left) Percent of CD62L+CD8+HA-CAR T cells grown in culture media containing indicated concentration of glucose and/or inosine from day 4 to day 21 and measured by flow cytometry. Data are mean±s.e.m. of n=5-8 donors. P values determined by paired two-tailed t-tests. (Right) HA-CAR T cells were grown in RPMI containing 11 mM of glucose and increasing concentrations of inosine for 17 days. Scatter plot showing correlation between CD62L frequency (y-axis) and inosine concentration (x-axis). Pearson correlation, r, with P values determined by paired two-tailed t-tests. N=2-6 donors from independent experiments.

FIG. 7D: Relative frequency of terminally differentiated effector (TEMRA; CD45RA+CD62L-), stem cell memory (CD45RA+CD62L+), central memory (CM; CD45RA-CD62L+), and effector memory (EM; CD45RA-CD62L-) in CD8+HA-CAR T cells cultured in different media conditions at day 21 post-activation measured by flow cytometry. (Top) Dot plots of representative donor shown. (Bottom) Frequencies of different populations polled from n=5-9 donors from independent experiments. P values determined by paired two-tailed t-tests.

FIG. 7E: (Left) Fold increase of IFNγ release after 24 hrs of co-culture with Nalm6-GD2 by D14 HA-CAR T cells grown in culture media containing various concentrations of glucose and/or inosine and normalized to CAR T cells grown in control media. Data are mean±s.e.m. of n=2-4 donors. P values determined by paired two-tailed t-tests. (Right) HA-CAR T cells were grown in RPMI containing 11 mM of glucose and increasing concentrations of inosine for 10 days, and stimulated with Nalm6-GD2 for 24 hrs. Scatter plot showing correlation between IFNγ secretion (y-axis) and inosine concentration (x-axis). Pearson correlation, r, with P values determined by paired two-tailed t-tests. N=2-4 donors from independent experiments.

FIG. 7F: Improvement index calculation scheme.

FIG. 7G: Scatter plot showing correlation between improvement index (y-axis-in black) or proliferation (y-axis-in red) and inosine concentration (x-axis). Pearson correlation, r, with P values determined by paired two-tailed t-tests. N=2-6 donors from independent experiments.

FIG. 8A: Schematic of experimental design.

FIG. 8B: Expansion rate of GD2.bbz-CAR T cells between day 3 and day 6 post-activation of CAR T cells grown in AIM 5 containing indicated concentration of inosine. N=4 donors from independent experiments. P values determined by paired two-tailed t-tests.

FIG. 8C: Viability of GD2.bbz-CAR T cells cultured for 4 days in AIM 5 containing indicated concentration of inosine at day 6 post-activation. N=4 donors from independent experiments. P values determined by paired two-tailed t-tests.

FIG. 8D: GD2.bbz-CAR T cells grown in media containing indicated inosine concentration between day 3 and day 8 post-activation and stimulated with 143b or Nalm6-GD2 tumor. IFNγ secretion after 24 hrs of co-culture was measured. Data are mean+s.d. of duplicate or triplicate wells. Representative of n=2 donors shown. P values determined by unpaired two-tailed t-tests.

FIG. 9A-9Y: Inosine (INO) induces stemness and augments CAR T cell function in vivo. 9A: Schematic of the experimental design. 9B: PCA of bulk RNA-seq from HA-CAR T cells expanded in glucose vs. inosine containing media for 10 days. 9C: GSEA of HA-CAR T cells expanded as in (B) using the GSE23321 gene collection. 9D: Levels of expression of indicated purinergic pathway gene transcripts from bulk RNA samples. Data are mean+/−s.e.m. from n=3 donors. P values determined by paired two-tailed t-tests. *, P<0.05; **, P<0.01; ***, P<0.001; **** P<0.0001. 9E: Flow cytometry analysis of CD62L expression by CD8+ and CD4+HA-CAR T cells at day 21 post-activation. Histograms of representative donor (Left) and pooled data from n=8 donors shown (Right). P values determined by paired two-tailed t-tests. *, P<0.05; **, P<0.01; ***, P<0.001; ****, P<0.0001. 9F: UMAP plots of day 14 CD8+ (top) and CD4+ (bottom) HA-or HA-INO-CAR T cells analyzed using CyTOF. 9,000 or maximum of CD4+ CAR T cells from each condition from n=4 donors were organized by their combined expression of 5 exhaustion markers. Graphs represent median expression of indicated markers. P values determined by paired two-tailed t-tests. *, P<0.05; **, P<0.01; ***, P<0.001; ****, P<0.0001. 9G: Histograms showing GD2 antigen expressed by Nalm6-GD2 and 143b tumor lines with corresponding number of molecules per cell. 9H: Co-cultures of HA, HA-ADA-O/E-or HA grown in INO stimulated with Nalm6-GD2 (1:8 E: T) at day 14 post-activation. Tumor GFP fluorescence intensity was normalized to the first time point (duplicate or triplicate wells). Representative donor of n=2 donor shown. P values determined by two-way ANOVA with Dunnett's multiple comparisons test. *, P<0.05; **, P<0.01; ***, P<0.001; ****, P<0.0001. 91: IFNγ secretion by day 14 HA-CAR T cells and HA-INO-CAR T cells, 24 hrs post-stimulation with Nalm6-GD2 (left) or 143b (right) tumor lines. Error bars represent mean+SD of triplicate wells from one representative donor of n=1-2 donors. P values determined by unpaired two-tailed t-tests. *, P<0.05; **, P<0.01; ***, P<0.001; ****, P<0.0001. 9J: IL-2 release after 24h of CD19.4-1BBz-CAR T cells stimulated with Nalm6 tumor line alone or in co-culture with HA-or HA-INO-CAR T cells at 1:1:1 ratio. Data are mean+SD from triplicate wells. N=1 donor. *, P<0.05; **, P<0.01; *** P<0.001; ****, P<0.0001. 9K: Schematic of the experimental design. 9L: Frequency of CD8+CD62L+HA-CAR T cells cultured in INO-media starting from the indicated days post-activation and assessed by flow cytometry at day 21. Data polled from n=5 donors from independent experiments. P values determined by paired two-tailed t-tests *, P<0.05; **, P<0.01; ***, P<0.001; ****, P<0.0001. 9M: Co-cultures of HA, or HA-CAR T cells grown in INO media starting from indicated days post-activation and 143b tumor (1:1 E: T) starting at day 15 post-activation. Tumor GFP fluorescence intensity was normalized to the first time point (duplicate or triplicate wells). Representative donor of n=3 donor shown. P values determined by two-way ANOVA with Dunnett's multiple comparisons test. *, P<0.05; **, P<0.01; ***, P<0.001; ****, P<0.0001. 9N: Co-cultures of CD19, or CD19-CAR T cells grown in INO media starting from indicated days post-activation and Nalm6-GD2 (1:10 E: T) starting at day 15 post-activation. Tumor GFP fluorescence intensity was normalized to the first time point (duplicate or triplicate wells). Representative donor of n=2 donor shown. P values determined by two-way ANOVA with Dunnett's multiple comparisons test. *, P<0.05; **, P<0.01; ***, P<0.001; ****, P<0.0001. 90: Cell expansion of HA-CAR T cells grown in culture media containing indicated concentration of glucose and/or inosine from day 4 to day 10 post-activation. Data are mean±s.e.m. of n=3-5 donors. P values determined by paired two-tailed t-tests. *, P<0.05; **, P<0.01; ***, P<0.001; ****, P<0.0001. 9P: Percent of CD62L+CD8+HA-CAR T cells grown in culture media containing indicated concentration of glucose and/or inosine from day 4 to day 21 and measured by flow cytometry. Data are mean±s.e.m. of n=5-8 donors. P values determined by paired two-tailed t-tests. *, P<0.05; **, P<0.01; ***, P<0.001; ****, P<0.0001. 9Q: Relative frequency of terminally differentiated effector (TEMRA; CD45RA+CD62L-), stem cell memory (CD45RA+CD62L+), central memory (CM; CD45RA-CD62L+), and effector memory (EM; CD45RA-CD62L-) in CD8+HA-CAR T cells cultured in different media conditions at day 21 post-activation measured by flow cytometry. (Top) Dot plots of representative donor shown. (Bottom) Frequencies of different populations polled from n=12 donors from independent experiments. P values determined by paired two-tailed t-tests *, P<0.05; **, P<0.01; ***, P<0.001; ****, P<0.0001. 9R: Fold increase of IFNg release after 24 hrs of co-culture with Nalm6-GD2 by D14 HA-CAR T cells grown in culture media containing various concentrations of glucose and/or inosine and normalized to CAR T cells grown in control media. Data are mean±s.e.m. of n=2-4 donors. P values determined by paired two-tailed t-tests. *, P<0.05; **, P<0.01; ***, P<0.001; ****, P<0.0001. 9S: HA-or HA cultured in inosine only (HA-INO) or inosine and glucose (HA-INO-GLC) containing media were repeatedly challenged with Nalm6-GD2 at 1:1 effector: target ratio (n=1 donor). Tumor GFP fluorescence intensity was normalized to the first time point (duplicate or triplicate wells). Representative donor of n=3 donor shown. P values determined by two-way ANOVA with Dunnett's multiple comparisons test. *, P<0.05; **, P<0.01; ***, P<0.001; ****, P<0.0001. 9T: IL-2 and IFNγ secretion after the third tumor challenge (mean+s.d. of 3 wells). P values determined by unpaired two-tailed t-tests. *, P<0.05; **, P<0.01; ***, P<0.001; ****, P<0.0001. 9U: Expansion of HA-CAR T cells grown in RPMI containing 11 mM of glucose and increasing concentrations of inosine for 6 days. Scatter plot showing correlation between ratio of expansion (y-axis) and inosine concentration (x-axis). Pearson correlation, r, with P values determined by paired two-tailed t-tests. N=2-4 donors from independent experiments. 9V: HA-CAR T cells were grown in RPMI containing 11 mM of glucose and increasing concentrations of inosine for 17 days. Scatter plot showing correlation between CD62L frequency (y-axis) and inosine concentration (x-axis). Pearson correlation, r, with P values determined by paired two-tailed t-tests. N=2-6 donors from independent experiments. 9W: HA-CAR T cells were grown in RPMI containing 11 mM of glucose and increasing concentrations of inosine for 10 days, and stimulated with Nalm6-GD2 for 24 hrs. Scatter plot showing correlation between IFNγ secretion (y-axis) and inosine concentration (x-axis). Pearson correlation, r, with P values determined by paired two-tailed t-tests. N=2-4 donors from independent experiments. 9X: At day 10 post-activation HA-CAR T cells were collected and cell cycle state was evaluated using BrdU and 7AAD staining. (Top) representative dot plots shown from one donor (n=3 donors). (Bottom) Bar graphs represent percentage of apoptotic cells or in G0/G1, S and G2/M phase indicated for each media conditions. Data are mean+/−s.e.m. from n=3 donors. P values determined by paired two-tailed t-tests. *, P<0.05; **, P<0.01; ***, P<0.001; ****, P<0.0001. 9Y: At day 10 post-activation CD19.28z-CAR T cells were collected and cell cycle state was evaluated using BrdU and 7AAD staining. (Top) representative dot plots shown. (Bottom) Bar graphs represent percentage of apoptotic cells or in G0/G1, S and G2/M phase indicated for each media conditions. Data are mean+/−s.e.m. from n=2 donors.

FIG. 10: Ten times more circulating T cells were observed in the blood of mice 17 days after receiving the Her2-INO-CAR vs. control Her2-CAR T cells Concentration of total CD3+ T cells detected in blood of mice at day 17 post-CAR T cell injection. P values determined by unpaired two-tailed t-test with Welch's correction. *, P<0.05; **, P<0.01; ***, P<0.001; ****, P<0.0001.

FIG. 11A-11H: Exhausted CAR T cells expanded in inosine undergo metabolic changes. A: Pathway Enrichment Analysis of day 14 CAR-T cells using the Reactome pathway collection and DAVID algorithm. Fold enrichment, number of genes represented, and FDR are shown. B: Heat map of differentially expressed genes in HA vs. HA-INO CAR-T cells (padj<0.01). C: Graphic illustration of metabolic changes upon inosine addition into glucose-free culture media. Upregulated genes highlighted in (B) shown in blue and downregulated genes shown in grey. D: UMAP analysis of CD8+CD19.28z-, HA-or HA-INO-CAR T cells 14 days post-activation. Expression of 28 markers (Panel 1) or 24 markers (Panel 2) was analyzed by CyTOF. 9,000 of CD8⁺ CAR T cells from each condition and donor (n-3-4) were combined and colored by marker intensity. Graphs represent median expression of indicated markers expressed by CAR+CD8+ T cells. P values determined by paired two-tailed t-tests. *, P<0.05; **, P<0.01; ***, P<0.001; ****, P<0.0001. E and F: Seahorse analysis of mitochondrial fitness was performed at day 11 post-activation. (E) Oxygen consumption rate (OCR) before and after treatment with oligomycin (Oligo), FCCP, and rotenone and antimycin (R+A). Plot shows mean+s.e.m from 7-9 technical replicates from one representative donor (n=3 donors). (F) Basal OCR, Maximal OCR and Spare Respiratory Capacity (SRC) from one representative donor. P values determined by two-way ANOVA with Dunnett's multiple comparisons test or Mixed-effects model with Dunnett's multiple comparisons test. G: Seahorse analysis of mitochondrial fitness was performed at day 11 post-activation. Oxygen consumption rate (OCR) before and after treatment with oligomycin (Oligo), FCCP, and rotenone and antimycin (R+A) and corresponding Maximal OCR and Spare Respiratory. Plots show mean±s.e.m from 7-9 technical replicates from one representative donor (n=3-4 donors). P values determined by two-way ANOVA with Dunnett's multiple comparisons test or Mixed-effects model with Dunnett's multiple comparisons test. H: Size of cells cultured in different media and assessed at day 13 post-activation. Data are mean+/−s.e.m. from n=3 donors. P values determined by paired two-tailed t-tests. *, P<0.05; **, P<0.01; *** P<0.001; ****, P<0.0001.

FIG. 12A-12H: Inosine metabolism affects HA-CAR T cells epigenetic by increasing polyamines metabolism. A: Global chromatin accessibility profile of D14 CD8+HA- and HA-INO CAR T cells from three healthy individuals determined by ATACseq (p-value<0.001 and log 2 FC>1). B: PCA of ATACseq chromatin accessibility from HA-CAR T cells cultivated in glucose or inosine. C: Overlayed accessibility profiles in HA (black) and HA-INO-CAR T cells (green) in the TCF7, IL2, IL7R, CCR7 and CXCR3 loci at day 14 post-activation. Concatenated samples from n=3 donors. D: Top transcription factor motifs enrichment in HA-INO vs. HA-T cells ranked by HOMER analysis. E: Top 25 transcription factor motif deviation scores in HA-INO versus HA− by chromVAR analysis. F: Levels of expression of elF5a gene transcripts from bulk RNA samples. Data are mean+/−s.e.m. from n=3 donors. P values determined by paired two-tailed t-tests. *, P<0.05; **, P<0.01; ***, P<0.001; ****, P<0.0001. G: Graphic illustration of targeting polyamine-hypusine circuit by ciclopirox (CPX). H: Relative frequency of terminally differentiated effector (TEMRA; CD45RA+CD62L-), stem cell memory (CD45RA⁺ CD62L⁺), central memory (CM; CD45RA-CD62L+), and effector memory (EM; CD45RA-CD62L-) in CD8⁺ HA-or HA-INO-CAR T cells pre-cultured in the presence of 20 μM CPX for 24 h at day 16 post-activation and measured by flow cytometry. (Left) Dot plots of representative donor shown. (Right) Frequencies of different populations polled from n=2 donors.

FIG. 13A-13H: Clinical scale manufacturing of clinical grade GD2-CAR T cells using media containing inosine, improves CAR product quality. A: Schematic of large-scale manufacturing of GD2.bbz-CAR+ T cells in a semi-closed G-Rex system in glucose-or inosine-containing media. 7 days post-activation, transduction efficiency, percentage of viable cells, and CD4/CD8 ratio of GD2+ cells were assessed by flow cytometry. Data are mean+/−s.e.m. from n=2 donors. P values determined by paired two-tailed t-tests. *, P<0.05; **, P<0.01; ***, P<0.001; ****, P<0.0001. B: Expansion of total viable T cells manufactured in control (black) vs. inosine (green)-containing media and (Right) corresponding number of CAR+ cells at day 7. Data are mean+/−s.e.m. from n=2 donors. P values determined by paired two-tailed t-tests. *, P<0.05; **, P<0.01; ***, P<0.001; ****, P<0.0001. C: IL-2 and IFNg secretion by CAR T cells stimulated for 24 hrs with 143b, mg63.3 or Nalm6-GD2 tumor lines. Error bars represent mean+SD of triplicate wells from one representative donor (n=2 donors). P values determined by unpaired two-tailed t-tests. *, P<0.05; **, P<0.01; ***, P<0.001; ****, P<0.0001. D: NSG mice were injected with 1×10⁶ Nalm6 leukemia cells. On day 3 post-tumor injection, 2×10⁶ of mock or GD2.bbz-CAR T cells manufactured in the presence of inosine or in regular media were transferred intravenously. Tumor growth was monitored by bioluminescent imaging. Data are mean±s.e.m. of n=5 mice per group. P values determined at day 43 by using Mann-Whitney test. Representative results of two independent experiments shown. *, P<0.05; **, P<0.01; *** P<0.001; ****, P<0.0001. E: Concentration of CD45+ T cells detected in blood of mice at D33 post-CAR T cell injection. P values determined by unpaired two-tailed t-test with Welch's correction. Results from one experiment shown. *, P<0.05; **, P<0.01; ***, P<0.001; ****, P<0.0001. F: Schematic of the experimental design. G: GD2.bbz-CAR T cells grown in media containing indicated inosine concentration between day 3 and day 10 post-activation and stimulated with 143b tumor. IL-2 and IFNg secretion after 24 hrs of co-culture was measured. Data are mean±s.d. of duplicate or triplicate wells. Representative of n=3 donors shown. P values determined by unpaired two-tailed t-tests. *, P<0.05; **, P<0.01; ***, P<0.001; ****, P<0.0001. H: Proposed model showing metabolic and epigenetic differences between tonic signaling CAR-T cells expanded in glucose-vs. inosine-containing culture media. Reprograming is due to an increase in glutaminolysis and polyamine synthesis and downregulation of CD73, FOXP3, A2aR and c-AMP response transcription factors. This results in an increase of stemness and a reduction of the immunosuppressive phenotype.

### DETAILED DESCRIPTION

Before the methods and compositions of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions. The upper and lower limits of these smaller ranges may independently be included in the smaller ranges and are also encompassed within the methods and compositions, subject to any specifically excluded limit in the stated range. Where the stated range includes one or both of the limits, ranges excluding either or both of those included limits are also included in the methods and compositions.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and compositions belong. Although any methods and compositions similar or equivalent to those described herein can also be used in the practice or testing of the methods and compositions, representative illustrative methods and compositions are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and compositions are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and compositions, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and compositions and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Manufacturing Therapeutic Cells

The present disclosure provides methods of manufacturing therapeutic cells. The methods comprise expanding cells in the presence of inosine, and harvesting the cells after expansion. The methods of the present disclosure are based in part on the inventors' unexpected findings that expanding cells in the presence of inosine (and even in the absence of glucose) results in the cells having: increased effector function both in vitro and in vivo; increased anti-tumor function in vivo; and increased percentage of memory/naïve cells; as compared to cells expanded under the same conditions but in the presence of glucose and in the absence of inosine. In addition, the present disclosure demonstrates that the cells may be expanded in the presence of inosine on a scale sufficient for clinical trials and therapeutic use. As proof of principle, this effect of inosine was initially tested in the context of CAR T cells. As will be appreciated with the benefit of the present disclosure, the methods find use in producing/manufacturing therapeutic cells (e.g., non-engineered T cells (e.g., tumor infiltrating lymphocytes (TILs)), T cells that express a recombinant T cell receptor (TCR), CAR T cells, CAR NK cells, and the like) with improved effector function for use in a wide variety of cell-based therapies, including but not limited to, cell-based therapies for treating cancer, e.g., solid tumors, hematological malignancies, etc. Details of the methods of the present disclosure will now be described.

As summarized above, the methods of the present disclosure comprise expanding cells in the presence of inosine. By “expanding” is meant the cells are cultured under conditions in which the cells proliferate. Suitable conditions may vary depending upon, e.g., the type of cells being expanded. Such conditions may include culturing the cell in a suitable container (e.g., a cell culture plate or well thereof, a cassette, tube, bottle or bag suitable for use in an automated therapeutic cell manufacturing system, e.g., a closed automated therapeutic cell manufacturing system such as the CliniMACS Prodigy® system by Miltenyi Biotec, the Xuri® cell expansion system by Cytiva, the G-Rex@ cell expansion system by Wilson Wolf, the Quantum® cell expansion system from Terumo, the Cocoon® system by Lonza, or the like), in suitable medium (e.g., cell culture medium, such as RPMI, DMEM, IMDM, MEM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%.

According to the methods of the present disclosure, the cell culture conditions include at least a period of time during which the cells are cultured in the presence of inosine. Inosine (C₁₀H₁₂N₄O₅) is a nucleoside that is formed when hypoxanthine is attached to a ribose ring (also known as a ribofuranose) via a β-N9-glycosidic bond. Inosine has the following structure:

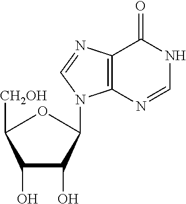

As used herein, the term “inosine” also includes analogs of inosine capable of functionally replacing inosine as an input into the central carbon metabolism (pentose phosphate pathway (PPP), glycolysis, and/or Krebs cycle) of the expanding cells.

In certain embodiments, expanding the cells in the presence of inosine comprises expanding the cells in a culture medium in which a suitable amount of inosine (in solid, e.g., powder, form) has been dissolved, or to which an appropriate volume of a concentrated solution of inosine has been added, to achieve a desired concentration of inosine in the culture medium. Inosine is available, e.g., from Sigma-Aldrich and elsewhere. In some embodiments, the cells are expanded in a culture medium comprising from 0.5 mM to 80 mM inosine, such as from 1 mM to 40 mM inosine, 2 mM to 20 mM inosine, 2 mM to 12 mM inosine (e.g., about 2.75 mM to about 11 mM inosine), 7 mM to 15 mM inosine, or 9 mM to 13 mM inosine, e.g., about 11 mM inosine. In some embodiments, the cells are expanded in a culture medium comprising from 2 mM to 12 mM inosine, e.g., about 2.75 mM to about 11 mM inosine.

Expanding the cells in the presence of inosine may commence at any desired time point. In certain embodiments, the cells are activated, and the cells are expanded in a culture medium supplemented with inosine at from 0 to 7 days post-activation. That is, the cells may be expanded in a culture medium comprising inosine commencing at the time of activation or commencing during expansion of the cells within 7 days post-activation. According to some embodiments, the cells are expanded in a culture medium supplemented with inosine at from 1 to 6 days post-activation, or from 2 to 5 days post-activation, e.g., from 3 to 4 days post-activation. As will be appreciated, peristaltic pumps may be implemented to move fluids and cells in and out of containers/bioreactors to transition the expansion of the cells from conditions in which inosine is absent to conditions in which inosine is present, and vice versa.

The cells are expanded in the presence of inosine for a desired period of time. According to some embodiments, the cells are expanded in the presence of inosine for from 12 hours to 14 days, such as from 2 to 12 days, 3 to 10 days, or 4 to 8 days.

As surprisingly demonstrated herein, cells may be expanded in the presence of inosine and in the absence of glucose (e.g., where inosine replaces glucose during the expansion), and cells manufactured under such conditions exhibit beneficial therapeutic properties as compared to cells expanded in the presence of glucose and in the absence of inosine, e.g., increased effector function both in vitro and in vivo, increased anti-tumor function in vivo, and increased percentage of memory/naïve cells. In certain embodiments, the cells are expanded in the presence of inosine in a culture medium that comprises less than 15 mM glucose, less than 14 mM glucose, less than 13 mM glucose, less than 12 mM glucose, less than 11 mM glucose, less than 10 mM glucose, less than 9 mM glucose, less than 8 mM glucose, less than 7 mM glucose, less than 6 mM glucose, less than 5 mM glucose, less than 4 mM glucose, less than 3 mM glucose, less than 2 mM glucose, or less than 1 mM glucose. According to some embodiments, the cells are expanded in the presence of inosine in a culture medium that comprises no glucose.

Methods for activating and expanding cells for therapy (e.g., therapeutic T cells and the like) are known in the art and are described, e.g., in U.S. Pat. Nos. 6,905,874; 6,867,041; and 6,797,514; and PCT Publication No. WO 2012/079000, the contents of which are hereby incorporated by reference in their entirety. In the example of T cells, such methods may include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is the Dynabeads® system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In certain embodiments, the cells are expanded using an automated system designed for the manufacture of therapeutic cells. Non-limiting examples of such systems include the CliniMACS Prodigy® system by Miltenyi Biotec, the Xuri® cell expansion system by Cytiva, the G-Rex® cell expansion system by Wilson Wolf, the Quantum® cell expansion system from Terumo, the Cocoon® system by Lonza, etc. Detailed guidance and protocols for manufacturing therapeutic cells on such systems are available from the providers of such systems.

The methods of the present disclosure result in the manufacture of therapeutic cells. By “therapeutic cells” is meant the cells are manufactured for use in a cell-based therapy. In some embodiments, the methods further comprise administering an effective amount of the manufactured cells or progeny thereof to a subject in need thereof as part of the cell-based therapy. As used herein, a “cell-based therapy” or “cell therapy” refers to the transfer of autologous or allogeneic cellular material into a subject/patient for medical purposes. Non-limiting examples of cell-based therapies include CAR T cell therapy, CAR NK cell therapy, engineered T cell therapy (e.g., T cells that express a recombinant T cell receptor (TCR)), a therapy comprising administering cells which do not express a recombinant receptor (e.g., tumor-infiltrating lymphocytes (TILs)), and the like.

The present methods find use in manufacturing a variety of types of therapeutic cells. In some embodiments, the therapeutic cells are immune cells. For example, the cells may be T cells, B cells, natural killer (NK) cells, macrophages, monocytes, neutrophils, dendritic cells, mast cells, basophils, eosinophils, or any combination thereof.

Thus, embodiments of the present methods include manufacturing therapeutic cells wherein the therapeutic cells are T cells. Examples of T cells include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17), CD4+ T cells, CD8+ T cells, virus-specific T cells, alpha beta T cells (Tαβ), and gamma delta T cells (Tγδ).

T cells may be obtained from any suitable source. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by reference in its entirety.

According to some embodiments, the present methods include manufacturing therapeutic cells wherein the therapeutic cells are stem cells, e.g., mammalian (e.g., human) stem cells. Non-limiting examples of stem cells include embryonic stem (ES) cells, adult stem cells, hematopoietic stem cells (HSCs), induced pluripotent stem cells (iPSCs), mesenchymal stem cells (MSCs), neural stem cells (NSCs), or any combination thereof.

In certain embodiments, prior to, during, or subsequent to expanding the cells in the presence of inosine, the cells are genetically modified. For example, according to some embodiments, the genetic modification comprises engineering the cells to express a receptor (e.g., a recombinant receptor) on the surface thereof.

A variety of suitable approaches for genetically modifying cells are available. A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

In order to express a desired polypeptide, a nucleotide sequence encoding the polypeptide can be inserted into appropriate vector, e.g., using recombinant DNA techniques known in the art. Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Illustrative examples of expression vectors include, but are not limited to pClneo vectors (Promega) for expression in mammalian cells; pLenti4/V 5-DEST™, pLenti6/V 5-DEST™, murine stem cell virus (MSCV), MSGV, moloney murine leukemia virus (MMLV), and pLenti6.2/V5-GW/lacZ (Invitrogen) for lentivirus-mediated gene transfer and expression in mammalian cells. In particular embodiments, a nucleic acid sequence encoding a polypeptide to be expressed in the cells may be ligated into such expression vectors for the expression of the polypeptides in mammalian cells.

Expression control sequences, control elements, or regulatory sequences present in an expression vector are those non-translated regions of the vector-origin of replication, selection cassettes, promoters, enhancers, translation initiation signals (Shine Dalgarno sequence or Kozak sequence), introns, a polyadenylation sequence, 5′ and 3′ untranslated regions, and/or the like-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

Components of the expression vector are operably linked such that they are in a relationship permitting them to function in their intended manner. In some embodiments, the term refers to a functional linkage between a nucleic acid expression control sequence (such as a promoter, and/or enhancer) and a second polynucleotide sequence, e.g., a nucleic acid encoding the polypeptide, where the expression control sequence directs transcription of the nucleic acid encoding the polypeptide.

In certain embodiments, the expression vector is an episomal vector or a vector that is maintained extrachromosomally. As used herein, the term “episomal” refers to a vector that is able to replicate without integration into the host cell's chromosomal DNA and without gradual loss from a dividing host cell also meaning that said vector replicates extrachromosomally or episomally. Such a vector may be engineered to harbor the sequence coding for the origin of DNA replication or “ori” from an alpha, beta, or gamma herpesvirus, an adenovirus, SV40, a bovine papilloma virus, a yeast, or the like. The host cell may include a viral replication transactivator protein that activates the replication. Alpha herpes viruses have a relatively short reproductive cycle, variable host range, efficiently destroy infected cells and establish latent infections primarily in sensory ganglia. Illustrative examples of alpha herpes viruses include HSV 1, HSV 2, and VZV. Beta herpesviruses have long reproductive cycles and a restricted host range. Infected cells often enlarge. Non-limiting examples of beta herpes viruses include CMV, HHV-6 and HHV-7. Gamma-herpesviruses are specific for either T or B lymphocytes, and latency is often demonstrated in lymphoid tissue. Illustrative examples of gamma herpes viruses include EBV and HHV-8.

According to some embodiments, the cell is engineered to express a chimeric antigen receptor (CAR), a T cell receptor (TCR) such as a recombinant TCR, a chimeric cytokine receptor (CCR), a chimeric chemokine receptor, a synthetic notch receptor (synNotch), a Modular Extracellular Sensor Architecture (MESA) receptor, a Tango receptor, a ChaCha receptor, a generalized extracellular molecule sensor (GEMS) receptor, a growth factor receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, an immunoreceptor tyrosine-based inhibition motif (ITIM)-containing receptor, a hormone receptor, a receptor tyrosine kinase, an immune receptor such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon-alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In certain embodiments, the cells are engineered to express a chimeric antigen receptor (CAR). According to some embodiments, the cell is engineered to express a recombinant TCR.

As described above, according to some embodiments, the cell may be engineered to express a CAR. The extracellular binding domain of the CAR may comprise a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). In some embodiments, the extracellular binding domain of the CAR is a single-chain version (e.g., an scFv version) of an antibody approved by the United States Food and Drug Administration and/or the European Medicines Agency (EMA) for use as a therapeutic antibody. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

When the cells are engineered to express a recombinant receptor on the surface thereof, the receptor may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a glycine-serine linker, such as a (G4S) 3 linker as described above. In certain embodiments, a linker separates one or more heavy or light chain variable domains, hinge domains, transmembrane domains, co-stimulatory domains, and/or primary signaling domains. In particular embodiments, the receptor (e.g., CAR) includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, when the cells are engineered to express a recombinant receptor on the surface thereof, the antigen binding domain of the receptor (e.g., CAR) is followed by one or more spacer domains that moves the antigen binding domain away from the cell surface (e.g., the surface of a T cell (e.g., a CD8+ or CD4+ T cell) expressing the receptor) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In some embodiments, the spacer domain includes the CH2 and/or CH3 of lgG1, lgG4, or IgD. Illustrative spacer domains suitable for use in the receptors (e.g., CARs) described herein include the hinge region derived from the extracellular regions of type 1 membrane proteins such as CD8a and CD4, which may be wild-type hinge regions from these molecules or variants thereof. In certain embodiments, the hinge domain includes a CD8a hinge region. According to some embodiments, the hinge is a PD-1 hinge or CD152 hinge. In certain embodiments, the hinge is an lgG4 hinge.

The “transmembrane domain” (Tm domain) is the portion of the receptor (e.g., CAR) that fuses the extracellular binding portion and intracellular signaling domain and anchors the receptor to the plasma membrane of the cell (e.g., T-cell, such as a Treg). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD33, CD3γ, CD30, CD4, CD5, CD8a, CD9, CD16, CD22, CD27, CD28, CD33, CD37, CD45, CD64, CD80, CD86, CD134, CD137, CD152, CD154, or PD-1.

In one embodiment, a receptor (e.g., CAR) includes a Tm domain derived from CD28. In certain embodiments, a receptor includes a Tm domain derived from CD28 and a short oligo-or polypeptide linker, e.g., between 1, 2, 3, 4, 5, 6, 7, 8, 9, or 10 amino acids in length, that links the Tm domain and the intracellular signaling domain of the receptor. A glycine-serine linker may be employed as such a linker, for example.

The “intracellular signaling” domain of a receptor (e.g., a CAR) refers to the part of the receptor that participates in transducing the signal from binding to a target molecule/antigen into the interior of the cell to elicit cell function. Accordingly, the term “intracellular signaling domain” refers to the portion of a protein which transduces the signal and that directs the cell to perform a specialized function. To the extent that a truncated portion of an intracellular signaling domain is used, such truncated portion may be used in place of a full-length intracellular signaling domain as long as it transduces the signal. The term intracellular signaling domain is meant to include any truncated portion of an intracellular signaling domain sufficient for transducing signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a receptor (e.g., CAR) expressed by a genetically modified cell may include an intracellular signaling domain that includes one or more (e.g., 1, 2, or more) “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a receptor of the present disclosure include those derived from FcRγ, FcRβ, CD3γ, CD3δ, CD3ε, CD3ζ, CD22, CD79α, CD79β, and CD66δ. In certain embodiments, a receptor includes a CD32 primary signaling domain and one or more costimulatory signaling domains. The intracellular primary signaling and costimulatory signaling domains are operably linked to the carboxyl terminus of the transmembrane domain.

In some embodiments, when the methods of the present disclosure are performed on cells engineered to express a recombinant receptor on the surface thereof, the receptor (e.g., CAR) includes one or more costimulatory signaling domains to enhance the efficacy and expansion of immune effector cells (e.g., T cells) expressing the receptor. As used herein, the term “costimulatory signaling domain” or “costimulatory domain” refers to an intracellular signaling domain of a costimulatory molecule or an active fragment thereof. Example costimulatory molecules suitable for use in receptors contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the receptor (e.g., CAR) includes one or more costimulatory signaling domains selected from the group consisting of 4-1BB (CD137), CD28, and CD134, and a CD37 primary signaling domain.

A receptor (e.g., CAR) expressed by a cell genetically modified according to the methods of the present disclosure may include any variety of suitable domains including but not limited to a leader sequence; hinge, spacer and/or linker domain(s); transmembrane domain(s); costimulatory domain(s); signaling domain(s) (e.g., CD3 domain(s)); ribosomal skip element(s); restriction enzyme sequence(s); reporter protein domains; and/or the like.

According to some embodiments, when the cells are genetically modified to express a receptor (e.g., a CAR) on their surface, the extracellular binding domain of the receptor specifically binds a tumor antigen expressed on the surface of a cancer cell. Non-limiting examples of tumor antigens to which the extracellular binding domain of the receptor may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvlll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside (“GD2”), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), Wilms' tumor 1 (WT1), and VEGF-A.

In certain embodiments, the cells are genetically modified to express an antibody. The term “antibody” (also used interchangeably with “immunoglobulin”) encompasses antibodies of any isotype (e.g., lgG (e.g., lgG1, lgG2, lgG3, or lgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the antigen, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein.

Immunoglobulin polypeptides include the kappa and lambda light chains and the alpha, gamma (lgG1, lgG2, lgG3, lgG4), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information system®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, lgG, IgM, IgA, IgD and IgE, respectively. In some embodiments, an antibody of the present disclosure is an lgG antibody, e.g., an lgG1 antibody, such as a human IgG1 antibody. In some embodiments, the cell expresses an antibody that comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Once the cells have been expanded to the desired extent, the methods comprise harvesting the cells, i.e., removing the cells from the container(s)/bioreactor(s) in which the cells were expanding at the time of harvest. Prior to or subsequent to harvesting the cells, the cells may be concentrated if desired, e.g., by centrifugation, a suitable cell separation technique (e.g., magnetic beads), and/or the like.

In some embodiments, the harvested cells are cryopreserved. As used herein, “cryopreserved” refers to cells that have been preserved or maintained by cooling to low sub-zero temperatures, such as 77 K or-196 deg. C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. Nos. 10,370,638; 10,159,244; 9078430; 7604929; 6136525; 5795711, the disclosures of which are incorporated herein by reference in their entirety. In contrast, the term “fresh”, as used herein with reference to cells, may refer to cells that have not been cryopreserved and, e.g., may have been directly obtained and/or used (e.g., transplanted, cultured, etc.) following collection from a subject or organ thereof.

In some embodiments, for cryopreservation, a cell suspension is aliquoted into one or more vessels and pelleted by centrifugation. Cell pellets may then be resuspended in cryopreservation media under cold conditions to reach a desired final concentration, such as e.g., million live cells per mL, and the resuspended cells kept at 4-8 deg. C. Cells prepared for cryopreservation may then be aliquoted into freezing containers and frozen using a controlled rate freezer. After controlled rate freezing is complete, cryopreserved may then be transferred to vapor phase liquid nitrogen for storage.

### Cells and Compositions

Aspects of the present disclosure further include cells and compositions. For example, in certain aspects, provided is a population of therapeutic cells manufactured according to the methods of manufacturing therapeutic cells of the present disclosure. Also provided are compositions comprising such cell populations.

Harvested therapeutic cell populations produced by the methods as described herein and therapeutic or pharmaceutical compositions thereof may be present in any suitable container (e.g., a culture vessel, tube, flask, vial, cryovial, cryo-bag, etc.) and may be employed (e.g., administered to a subject) using any suitable delivery method and/or device. Such populations of cells and pharmaceutical compositions may be prepared and/or used fresh or may be cryopreserved. In some instances, populations of therapeutic cells and pharmaceutical compositions thereof may be prepared in a “ready-to-use” format, including e.g., where the therapeutic cells are present in a suitable diluent and/or at a desired delivery concentration (e.g., in unit dosage form) or a concentration that can be readily diluted to a desired delivery concentration (e.g., with a suitable diluent or media). Populations of therapeutic cells and pharmaceutical compositions thereof may be prepared in a delivery device or a device compatible with a desired delivery mechanism or the desired route of delivery, such as but not limited to e.g., a syringe, an infusion bag, or the like.

In some instances, the present disclosure provides one or a plurality of cell therapy doses, e.g., each contained in suitable container. Cell therapy doses may be generated through a variety of methods. Aliquoting expanded populations of therapeutic cells into cell therapy doses may be performed by a variety of means. In some instances, a cell therapy dose includes, e.g., at least 10 million, at least 25 million, at least 50 million, at least 75 million, at least 100 million, at least 250 million, at least 500 million, at least 750 million, at least 1 billion, at least 2 billion, at least 3 billion, at least 4 billion, at least 5 billion, at least 6 billion, at least 7 billion, at least 8 billion, at least 9 billion, at least 10 billion, at least 15 billion, at least 20 billion, at least 30 billion, at least billion, at least 50 billion, at least 60 billion, at least 70 billion, at least 80 billion, at least 90 billion, or at least 100 billion therapeutic cells.

In certain embodiments, the compositions may include the therapeutic cells present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid sodium salt (MES), 3-(N-Morpholino) propanesulfonic acid (MOPS), N-tris [Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

The compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease or disorder associated, e.g., with the target cell or a population thereof, as compared to a control. An effective amount can be administered in one or more administrations.

A “therapeutically effective amount” of such cells may vary according to factors such as the disease state, age, sex, and weight of the subject, and the ability of the cells to elicit a desired response in the subject. A therapeutically effective amount is also one in which any toxic or detrimental effects of the cells are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject). In certain aspects, a pharmaceutical composition of the present disclosure includes from 1×10⁶ to 5×10¹⁰ of the cells produced according to the methods of the present disclosure.

The cells of the present disclosure can be incorporated into a variety of formulations for therapeutic administration. More particularly, the cells of the present disclosure can be formulated for administration by combination with appropriate excipients, diluents and/or the like.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

An aqueous formulation of the cells may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

In some embodiments, a composition includes cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/V).

### Methods of Administering Cell-Based Therapies

Aspects of the present disclosure further include methods of using a population of therapeutic cells manufactured according to the cell manufacturing methods described herein, which methods result in therapeutic cells (e.g., non-engineered T cells (e.g., tumor infiltrating lymphocytes (TILs)), T cells that express a recombinant T cell receptor (TCR), CAR T cells, CAR NK cells, and the like) having improved effector function for use in a wide variety of cell-based therapies, including but not limited to, cell-based therapies for treating cancer, e.g., solid tumors, hematological malignancies, etc. Accordingly, aspects of the present disclosure include methods comprising administering an effective amount of such a population of cells to a subject (e.g., a human subject) in need thereof.

The therapeutic cells may be autologous/autogeneic (“self”) or non-autologous (“non-self,” e.g., allogeneic, syngeneic or xenogeneic). “Autologous” as used herein, refers to cells obtained from the subject to whom the therapeutic cells are later administered. “Allogeneic” as used herein refers to cells obtained from a donor other than the subject to whom the therapeutic cells are administered. In some embodiments, the cells (e.g., T cells) are cells obtained from a mammalian subject. In certain embodiments, the mammalian subject is a primate. In some embodiments, the cells are obtained from a human.

Any of the cell-based therapeutic methods of the present disclosure may be used to treat a variety of conditions in the subject. In certain embodiments, the subject has cancer. The methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), diffuse midline glioma (DMG), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, squamous non-small cell lung cancer, rhabdomyosarcoma, metastatic renal cell carcinoma, basal cell carcinoma (basal cell epithelioma), and gliosarcoma. In some embodiments, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

In certain embodiments, the cancer comprises a solid tumor. According to some embodiments, the solid tumor is a carcinoma or a sarcoma. When the solid tumor is a carcinoma, in certain embodiments, the carcinoma is a basal cell carcinoma, squamous cell carcinoma, renal cell carcinoma, ductal carcinoma in situ (DCIS), invasive ductal carcinoma, or adenocarcinoma. By treatment is meant at least an amelioration of one or more symptoms associated with the condition of the subject, where amelioration is used in a broad sense to refer to at least a reduction in the magnitude of a parameter, e.g., symptom, associated with the condition being treated. As such, treatment also includes situations where the condition, or at least one or more symptoms associated therewith, are completely inhibited, e.g., prevented from happening, or stopped, e.g., terminated, such that the subject no longer suffers from the condition, or at least the symptoms that characterize the condition.

### Kits

Aspects of the present disclosure further include kits. In certain embodiments, the kits find use in practicing the therapeutic cell manufacturing methods of the present disclosure.

In some embodiments, provided are kits comprising inosine and instructions for expanding cells in a culture medium comprising the inosine. According to some embodiments, the inosine is present in a cell culture medium. Non-limiting examples of cell culture media that may be provided in a kit of the present disclosure include RPMI, DMEM, IMDM, MEM, DMEM/F-12, or the like.

According to some embodiments, a kit of the present disclosure comprises a cell culture medium, or comprises instructions for using the inosine to produce a cell culture medium, comprising from 0.5 mM to 80 mM inosine, such as from 1 mM to 40 mM inosine, 2 mM to 20 mM inosine, 7 mM to 15 mM inosine, or 9 mM to 13 mM inosine, e.g., about 11 mM inosine. In certain embodiments, a kit of the present disclosure comprises a cell culture medium, or comprises instructions for using the inosine to produce a cell culture medium, comprising less than 15 mM glucose, less than 14 mM glucose, less than 13 mM glucose, less than 12 mM glucose, less than 11 mM glucose, less than 10 mM glucose, less than 9 mM glucose, less than 8 mM glucose, less than 7 mM glucose, less than 6 mM glucose, less than 5 mM glucose, less than 4 mM glucose, less than 3 mM glucose, less than 2 mM glucose, less than 1 mM glucose, or no glucose.

In certain embodiments, a kit of the present disclosure includes one or more containers into which the expanded cells may be harvested, e.g., a tube, flask, vial, cryovial, cryo-bag, etc. According to some embodiments, a kit of the present disclosure comprises one or more reagents for cryopreserving the expanded cells.

Components of the kits may be present in separate containers, or multiple components may be present in a single container. A suitable container includes a single tube (e.g., vial), one or more wells of a plate (e.g., a 96-well plate, a 384-well plate, etc.), or the like.

The instructions of the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### Experimental

### Example 1-In Vitro Cell Expansion in the Presence of Inosine Increases Effector Function of Non-Exhausted CAR T Cells In Vitro

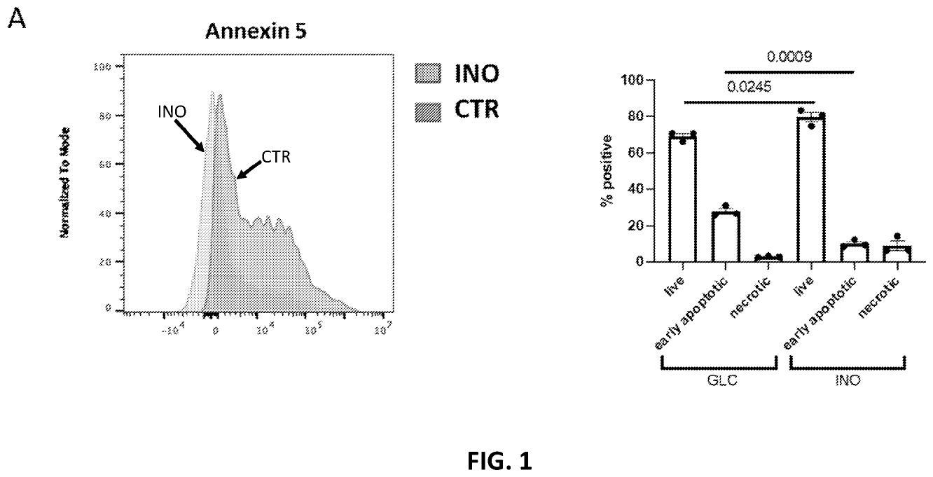

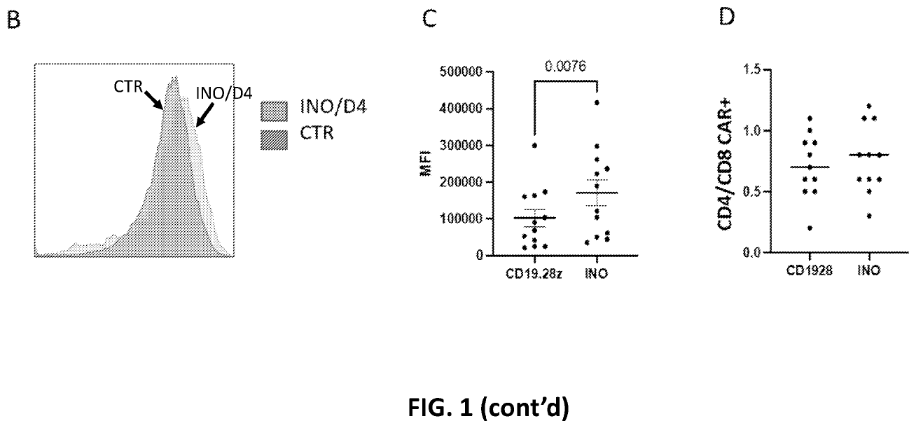

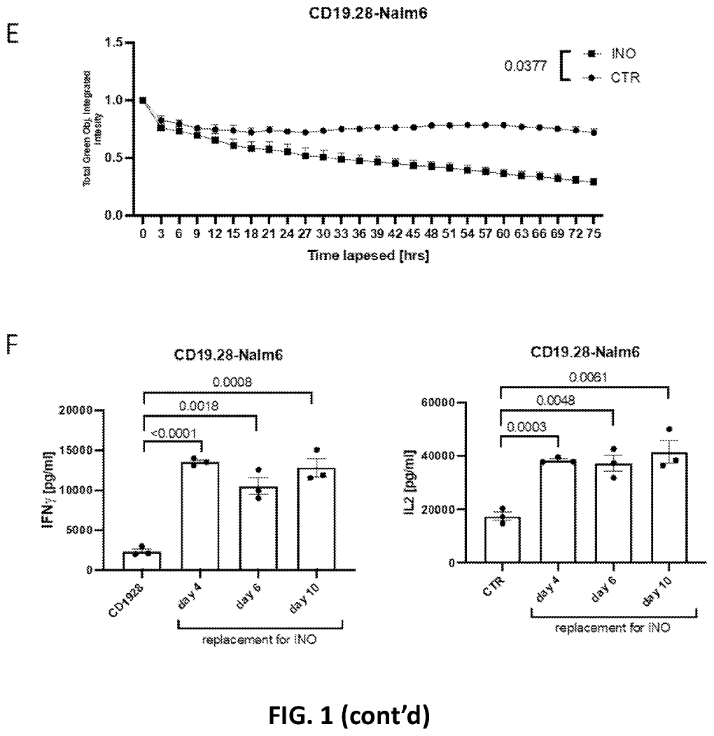

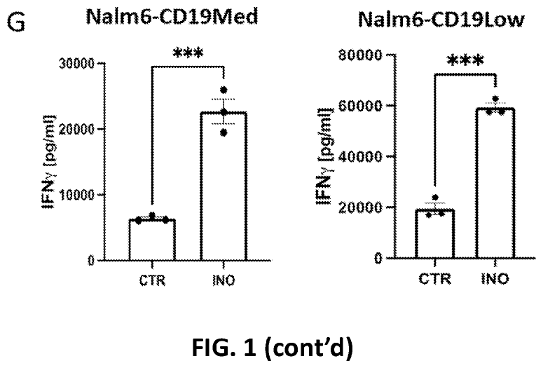

In this example, CD19.28 CAR T cells were generated using a retroviral vector and at day 4 post-activation, the cells were split between 11 mM glucose-(CTR) or 11 mM inosine-(INO) containing media (unless stated otherwise). Tested first was whether the absence of glucose in the culture media has a negative effect on the viability of CAR T cells. Surprisingly, not only was a significantly lower frequency of Annexin V+ apoptotic cells observed, but an increase in the percentage of live cells was also observed (FIG. 1A). Despite increased CAR MFI in the INO condition (FIG. 1C), neither percentage of CAR+ T cells nor CD4/CD8 ratio was affected by the presence of inosine (FIGS. 1B and 1D). Moreover, those cells exhibited increased tumor killing (FIG. 1E) and effector cytokine secretion (FIG. 1F) when stimulated with cells of the Nalm6 leukemia tumor line.

Tumor antigen density is one of the most important factors affecting the efficacy of CAR T immunotherapy. Therefore, assessed next was whether growing CAR T cells in inosine-containing media improves their effector function against low antigen expressing tumor lines. Surprisingly, when CD19.28 CAR T cells were stimulated with Nalm6 expressing medium or low levels of surface CD19, a significantly increased secretion of IFNγ was observed (FIG. 1G).

Taken together, the data in this example demonstrate that in vitro cell expansion in the presence of inosine increases effector function of non-exhausted CAR T cells in vitro.

### Example 2-In Vitro Cell Expansion in the Presence of Inosine Increases Effector Function of Exhausted CAR T Cells In Vitro

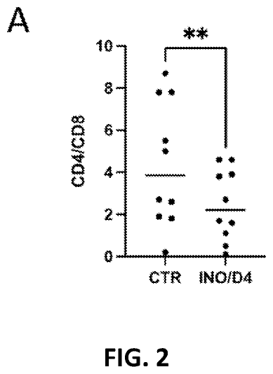

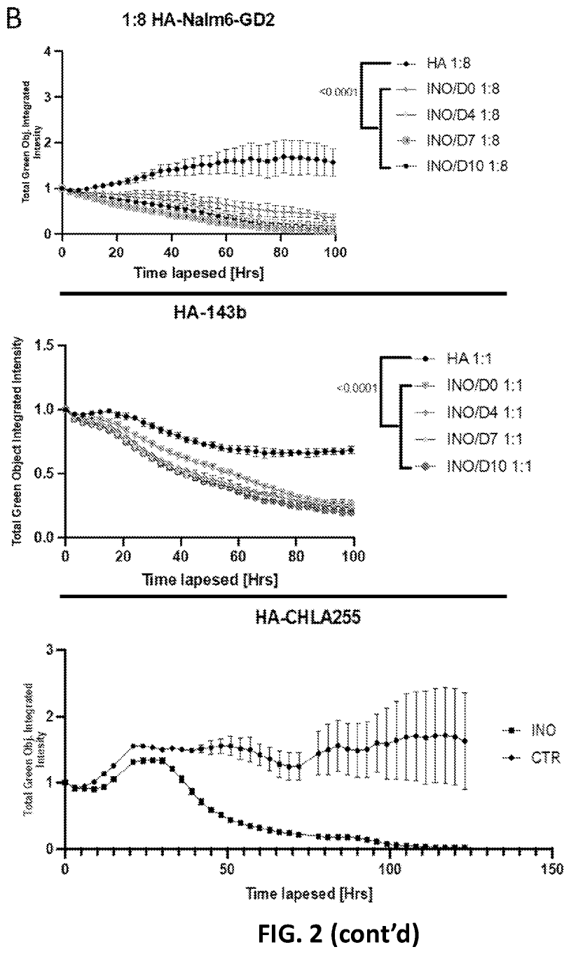

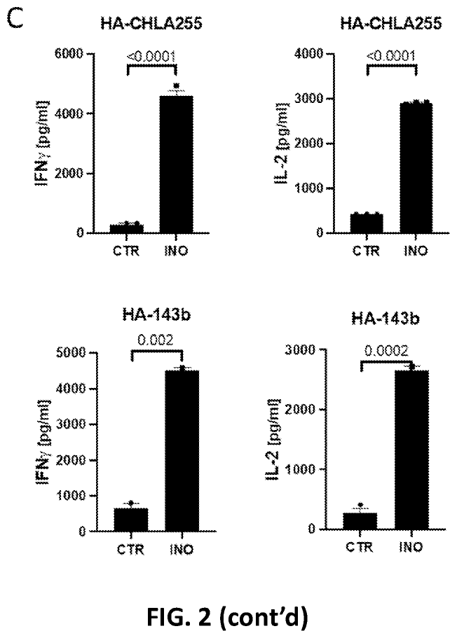

In this example, using an in vitro model of human T cell exhaustion induced by a tonically signaling CAR (HA), it was surprisingly found that expansion of exhausted CD8+ T cells in inosine-containing (INO+) media increased the frequency of CD8+ cells, as compared to the control group in which inosine was absent (FIG. 2A). HA-INO CAR T cells exhibited significantly increased cytotoxic function assessed by secretion of IL-2 and IFNγ, and IncuCyte killing, when stimulated with tumor cell lines expressing different levels of target antigen GD2 (FIGS. 2B and C).

Thus, the data in this example demonstrate that in vitro cell expansion in the presence of inosine increases effector function of exhausted CAR T cells in vitro.

### Example 3-Increased Inosine Metabolism Results in a Higher Frequency of Memory/Naïve And a Decreased Frequency of Terminally Differentiated CAR T Cells

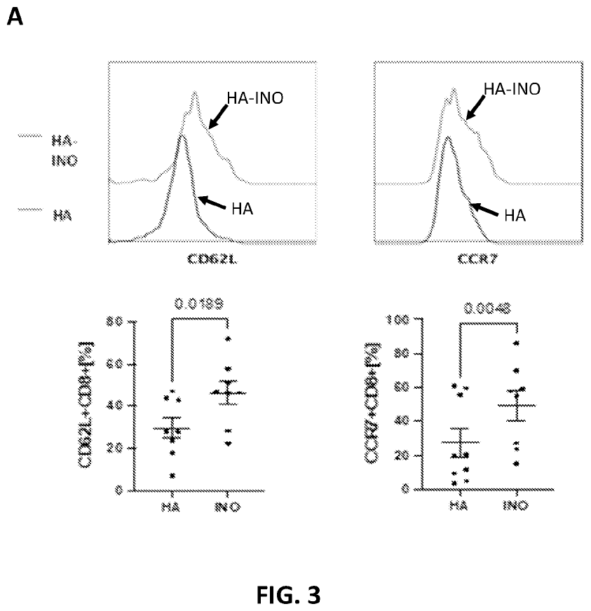

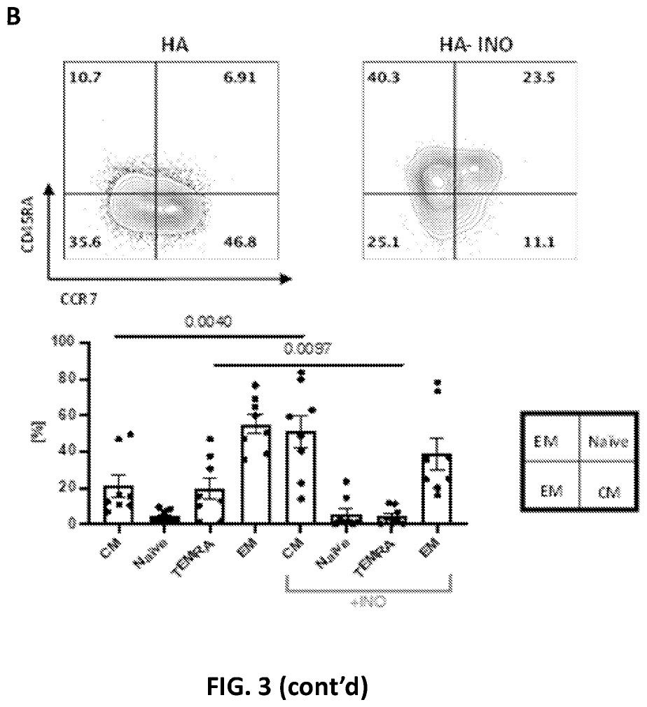

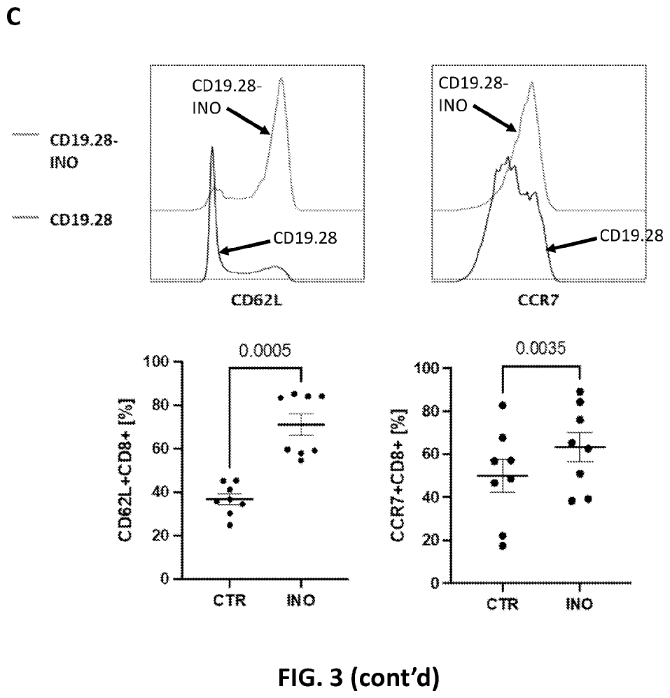

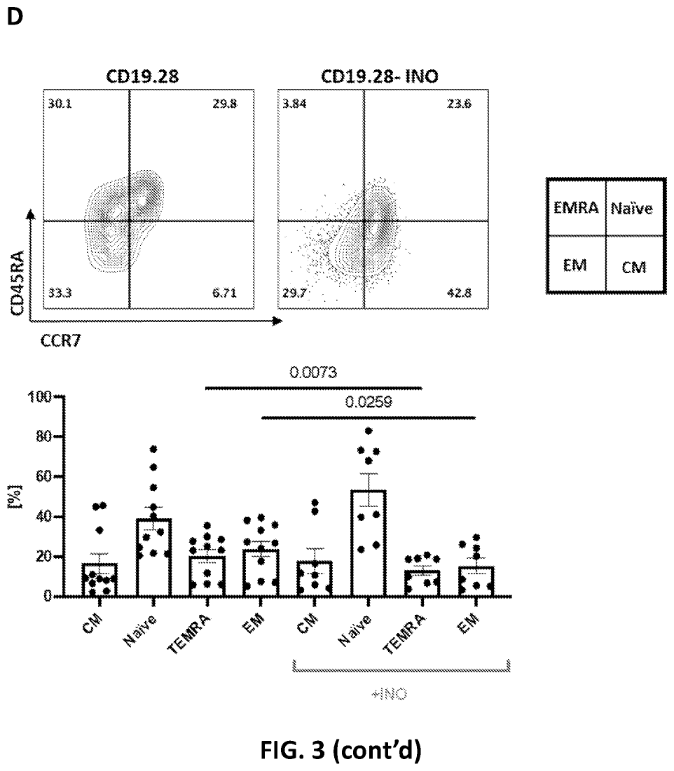

Demonstrated in this example is that both HA cultured in INO+ media exhibit higher expression of memory markers such as CD62L and CCR7 (FIG. 3A). More in-depth analysis of those cells revealed that inosine supplementation resulted in an elevated percentage of naïve and central memory HA CAR T cells and decreased frequency of terminally differentiated and effector cells (FIG. 3B). Similar results were observed in CD19.28 CAR T cells (FIGS. 3C and D).

### Example 4-Inosine Alters the Metabolic Phenotype of CAR T Cells

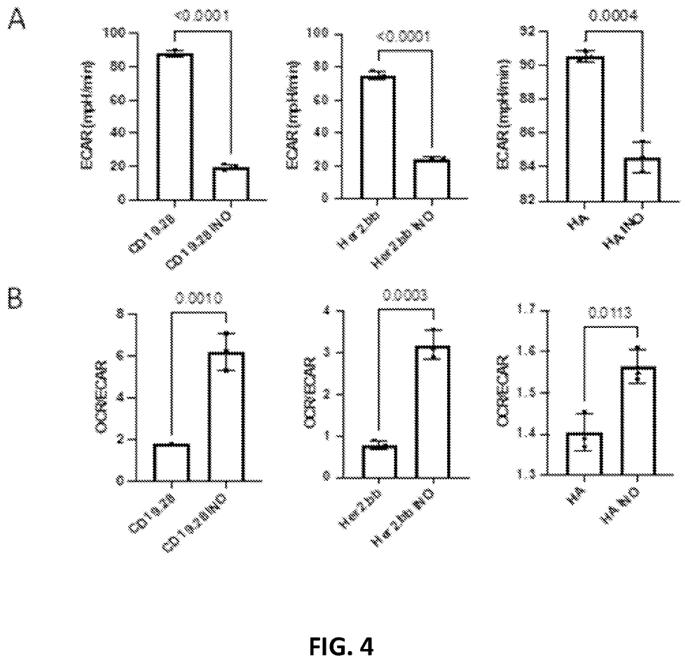

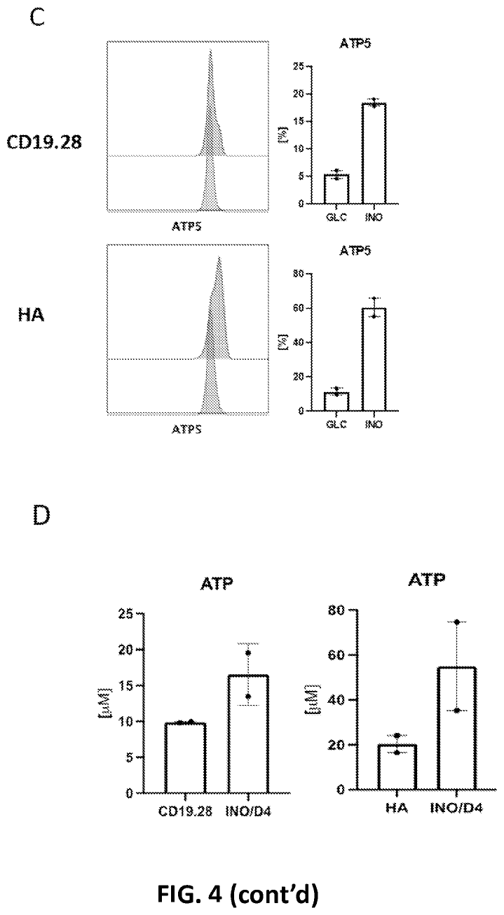

Studied next was the effect of inosine on CAR T cell metabolism. Seahorse analysis revealed that cells manufactured in the presence of inosine exhibit significantly decreased extracellular acidification rate (ECAR), a surrogate parameter for glycolysis (FIG. 4A). This decrease in the glycolytic flux resulted in a change in the cell's bioenergetic profile, shifting it towards oxidative phosphorylation (FIG. 4B). This correlated with an increased level of mitochondrial membrane ATP synthase (Complex V) ATP5 protein expression (FIG. 4C), a complex that is responsible for ATP production by mitochondria. Furthermore, the total amount of ATP generated by CAR T cells grown in the presence of inosine was increased (FIG. 4D).

Taken together, the data demonstrate that inosine supplementation induces phenotypic, metabolic and functional changes in exhausted and non-exhausted CAR T cells in vitro.

### Example 5-CAR T Cells Manufactured in Inosine-Containing Media Exhibit Increased Anti-Tumor Function In Vivo

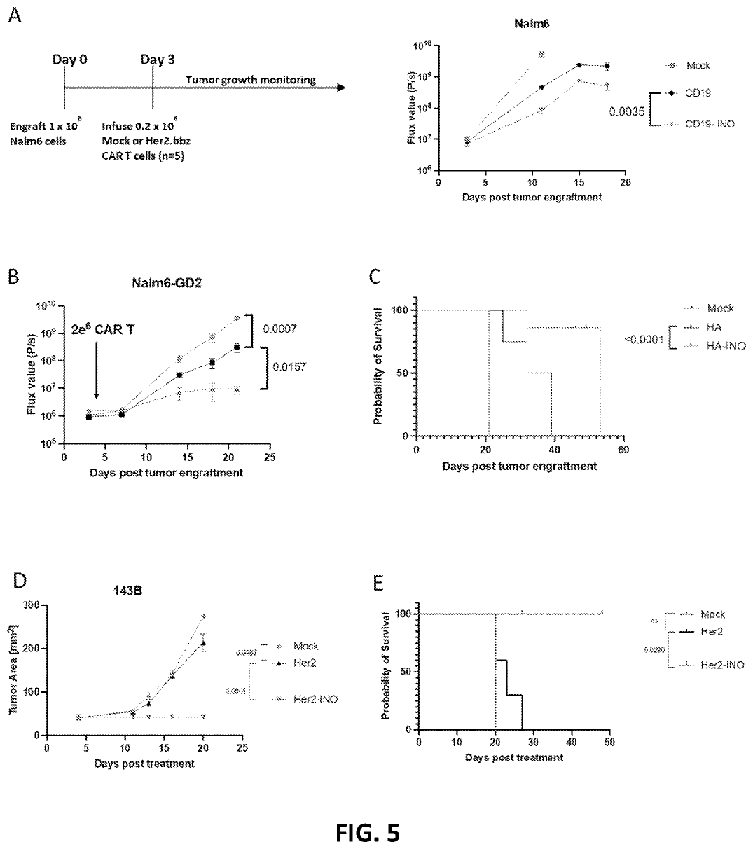

In this example, the in vivo anti-tumor potency of CAR T cells manufactured in the presence or absence of inosine was assessed. First, NSG mice were inoculated with 1×10⁶ Nalm6-firefly luciferase (fLuc) tumor cells intravenously (IV) on day 0. Three days post tumor injection, mice were evaluated and normalized for engraftment using bioluminescent imaging. Five mice per group were injected IV with 0.2×10⁶ CD19.28 CAR T cells on day 3. Weekly tumor bioluminescent imaging showed that CAR T cells manufactured in the presence of inosine significantly delayed tumor growth (FIG. 5A). Indeed, inosine-grown CAR T cells significantly delayed tumor growth in this “in vivo stress test”. Similar results were obtained when leukemia-bearing mice were injected with HA CAR T cells. Surprisingly, cells expanded in the presence of inosine exhibited improved tumor control and survival as compared to CAR T cells grown in regular media not containing inosine (FIGS. 5B and 5C).

In a solid tumor model, NSG mice were inoculated with 1×10⁶ of 143 osteosarcoma tumor via intramuscular injections. Four days later, 10×10⁶ Mock, Her2.bb cultured in regular media (Her2) or Her2.bb cultured in inosine-containing media (Her2-INO) CAR T cells were injected into the mice. Surprisingly, the group of mice injected with cells expanded in the presence of inosine (INO-CAR T cells) showed not only significantly lower tumor burden but also overall prolonged survival (FIGS. 5D and E).

Thus, CAR T cells manufactured in inosine-containing media exhibit increased anti-tumor function in vivo.

### Example 6-Large-Scale Manufacturing of Clinical GD2 CAR T Cells in a Semi-Closed System Using Inosine-Containing Media Improves CAR Product Quality

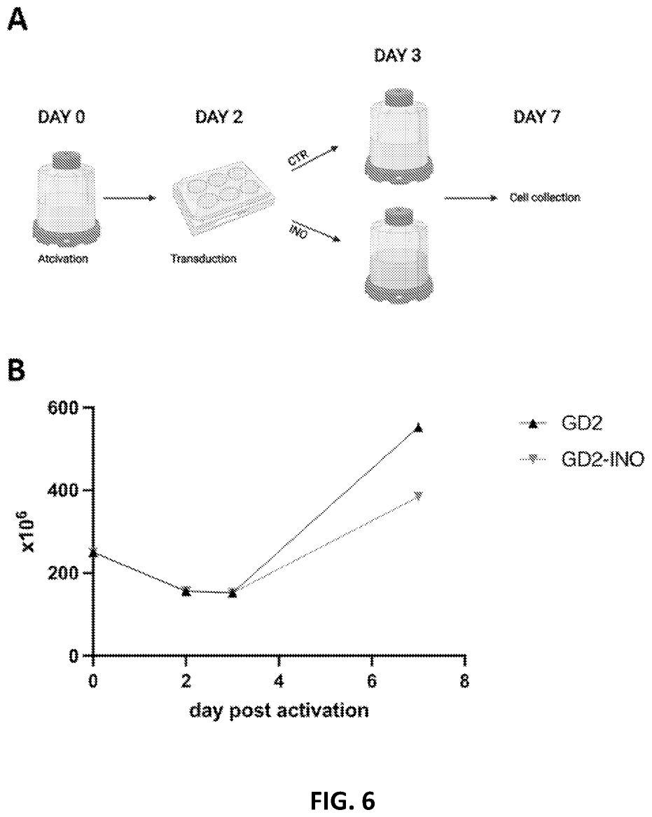

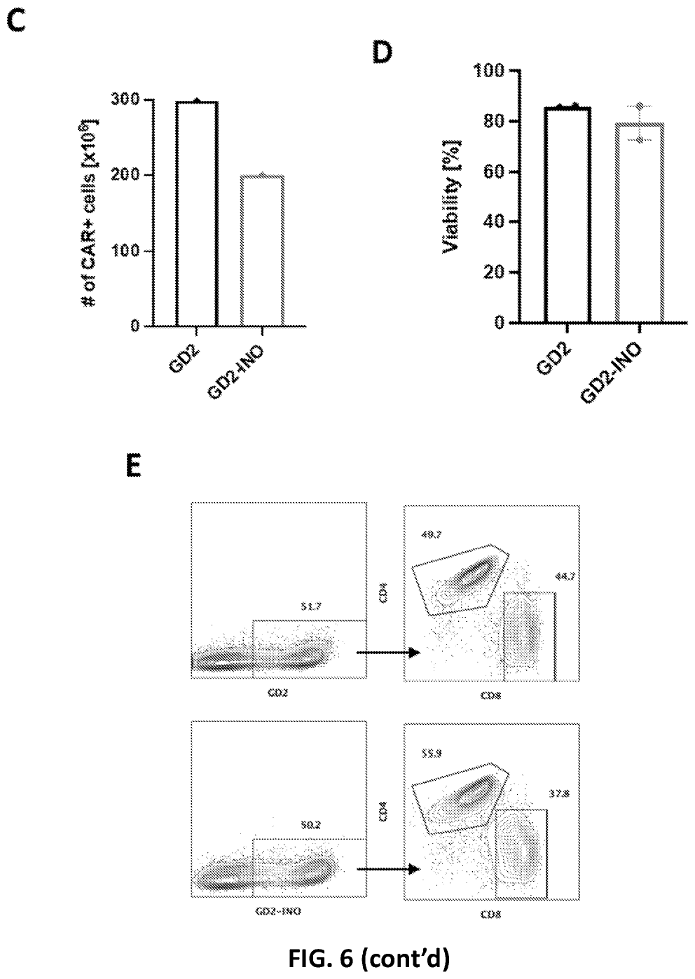

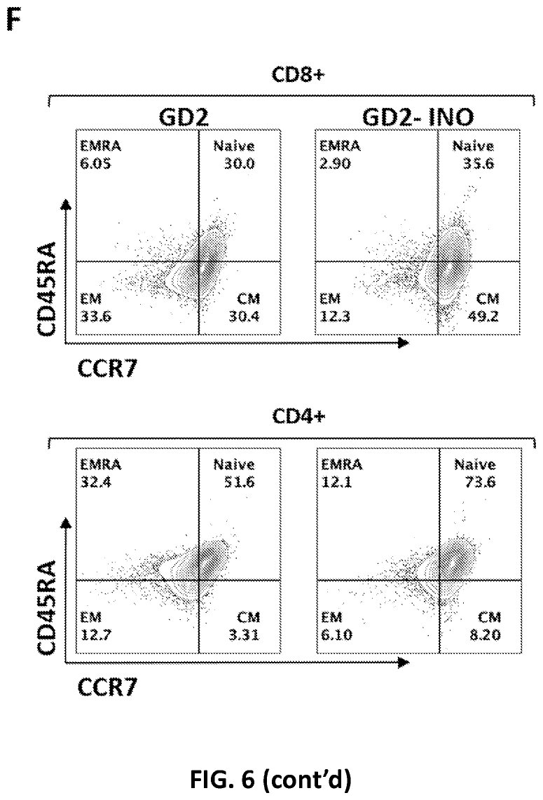

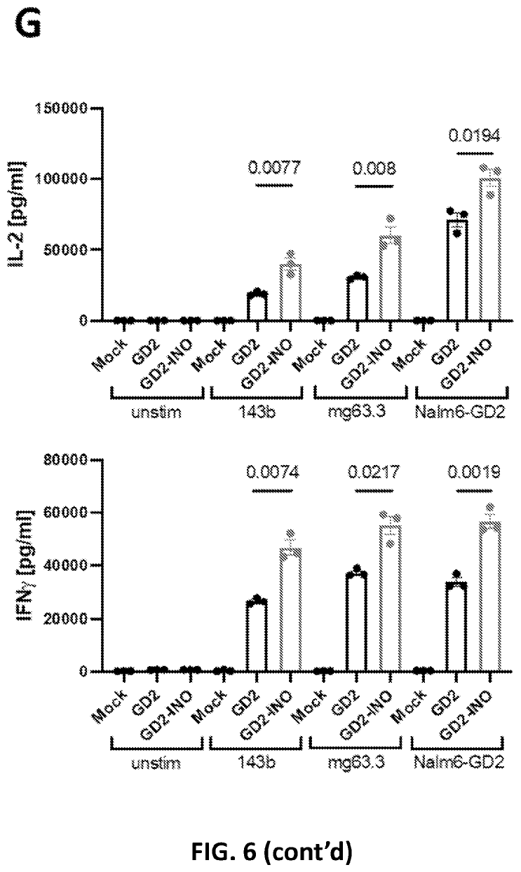

In this example, the in vivo anti-tumor potency of CAR T cells manufactured in the presence or absence of inosine was assessed. In this particular example, the feasibility of manufacturing GD2 CAR T cells in inosine media in the clinical setting was assessed. 2.5×10⁸ enriched CD4+ and CD8+ T cells from a healthy donor were activated with anti-CD3/CD28-coated beads and transduced using clinical grade GD2.bbz vector at MOI=10 (FIG. 6A). From day 3, T cells were continued to be cultured in regular-or inosine containing media in a semi-closed culture system using G-Rex platform. Although, inosine-manufactured cells proliferated at slower rate than the control (384.3×10⁶ vs 552.7×10⁶ total cells, respectively) (FIG. 6B), enough CAR T cells were able to be obtained to formulate dose level 1 (1×10⁶ CAR+ T cells/kg body weight (+/−20%)) (FIG. 6C). There was no difference in product viability (FIG. 6D), nor transduction efficiency nor CD4 vs CD8 ratio (FIG. 6E). Similarly, to the small-scale experiments, there was a higher frequency of naive and central memory GD2 CAR T cells, when cultured in the presence of inosine (FIG. 6F). Further, GD2-INO CAR T cells secreted significantly more both IL-2 and IFNγ when stimulated with 143b (low GD2 expression), mg63.3 or Nalm6-GD2 (high GD2 expression) (FIG. 6G).

This data demonstrates that CAR T cells can be successfully expanded in inosine-containing media to reach a necessary dose for clinical trials and therapeutic use.

### Example 7-Inosine Increases Stemness Features, Enhances T Cell Functionality, and Abrogates Immune Suppression in Chronically Activated CAR T Cells Independently of the Presence of Glucose

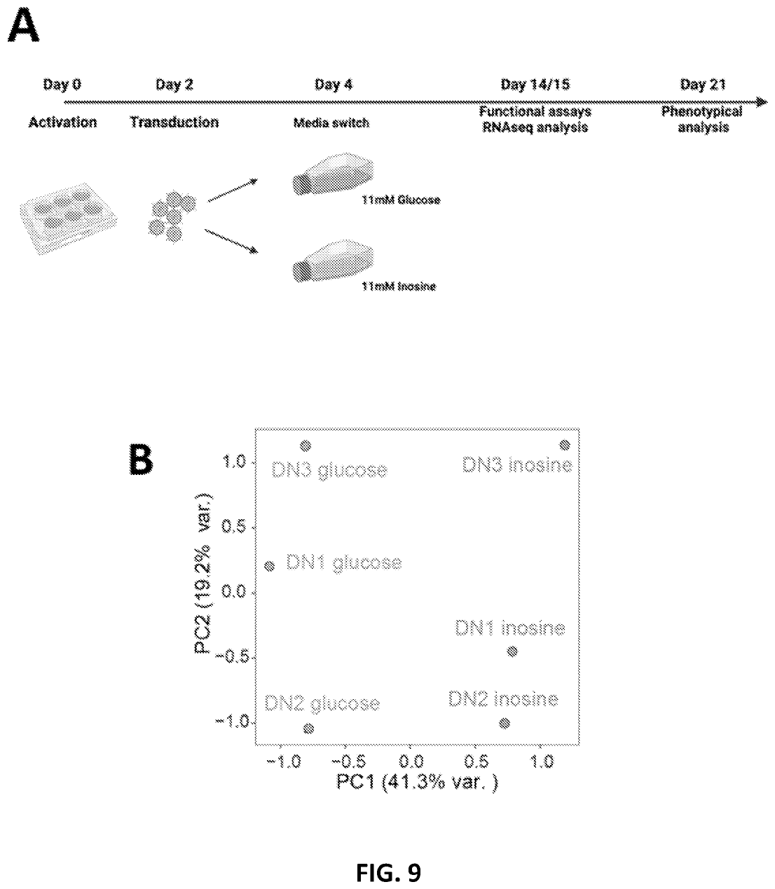

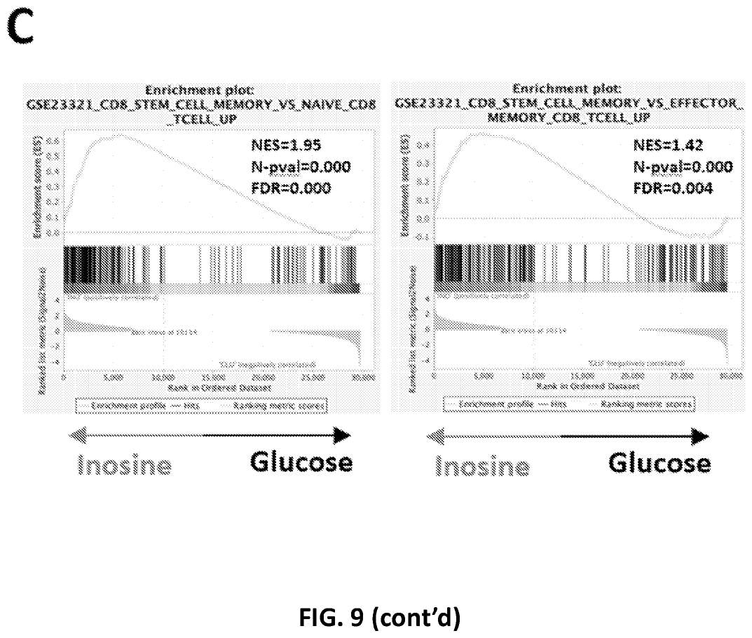

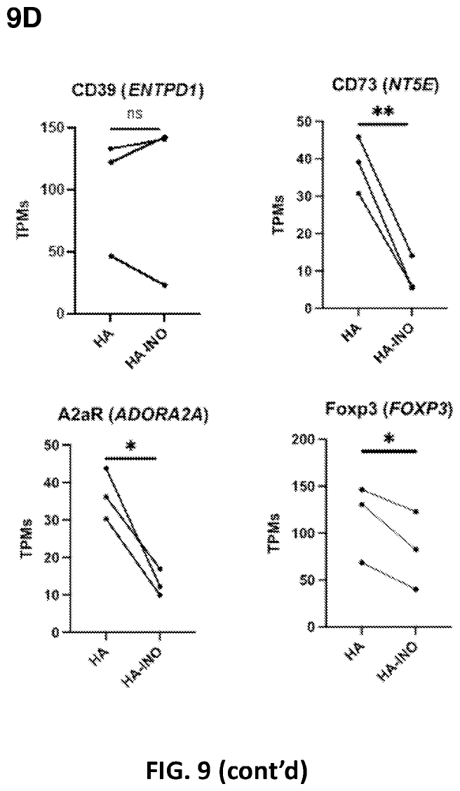

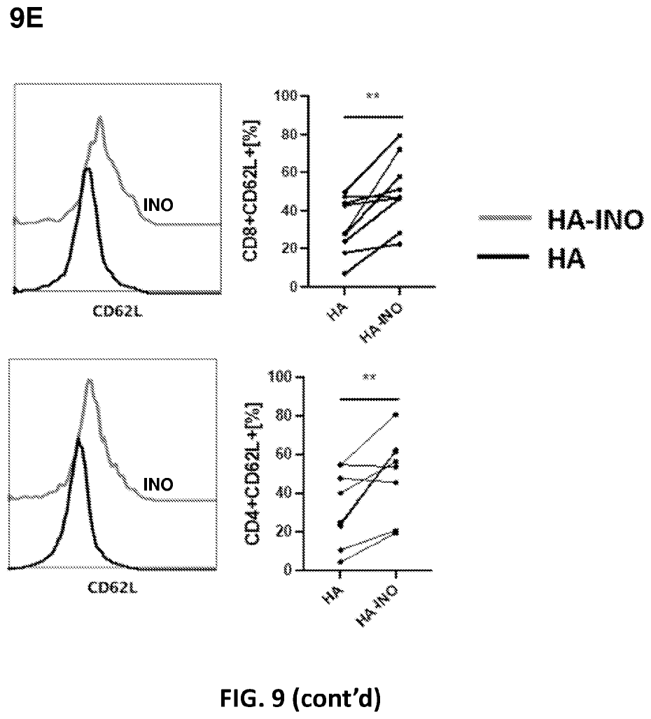

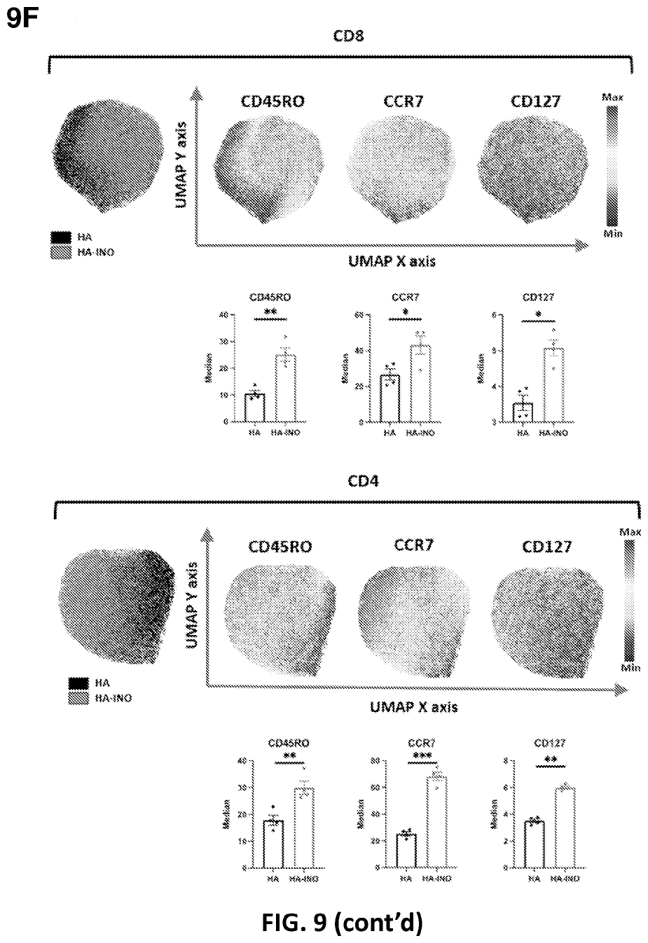

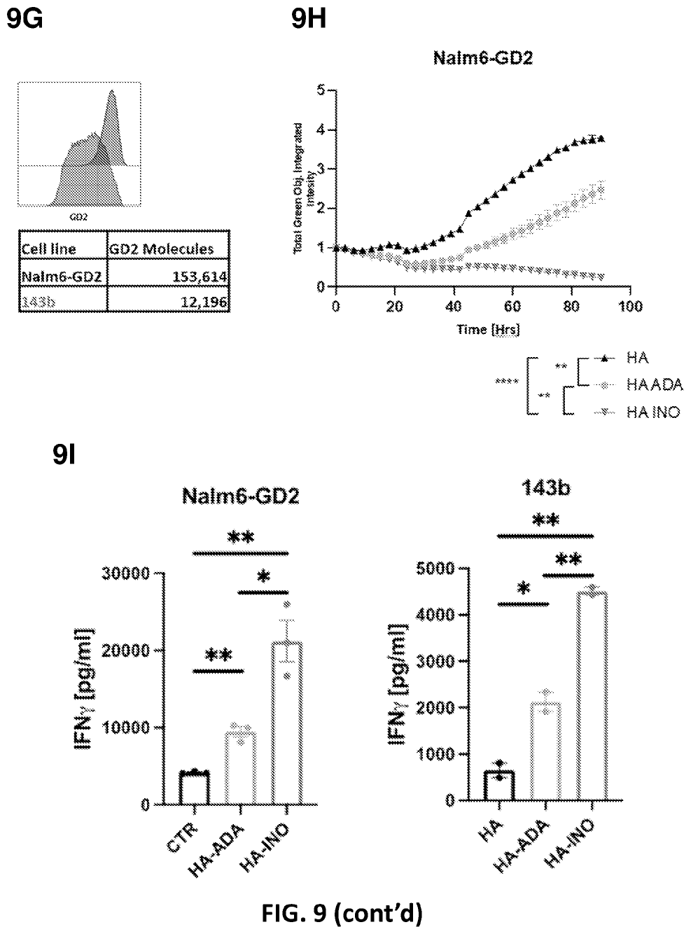

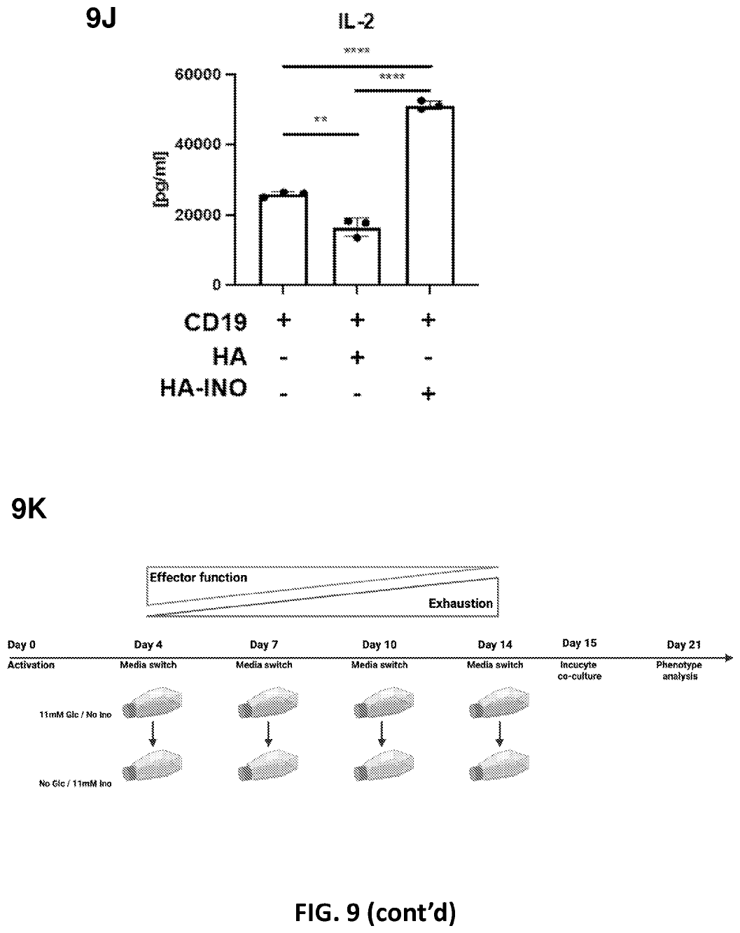

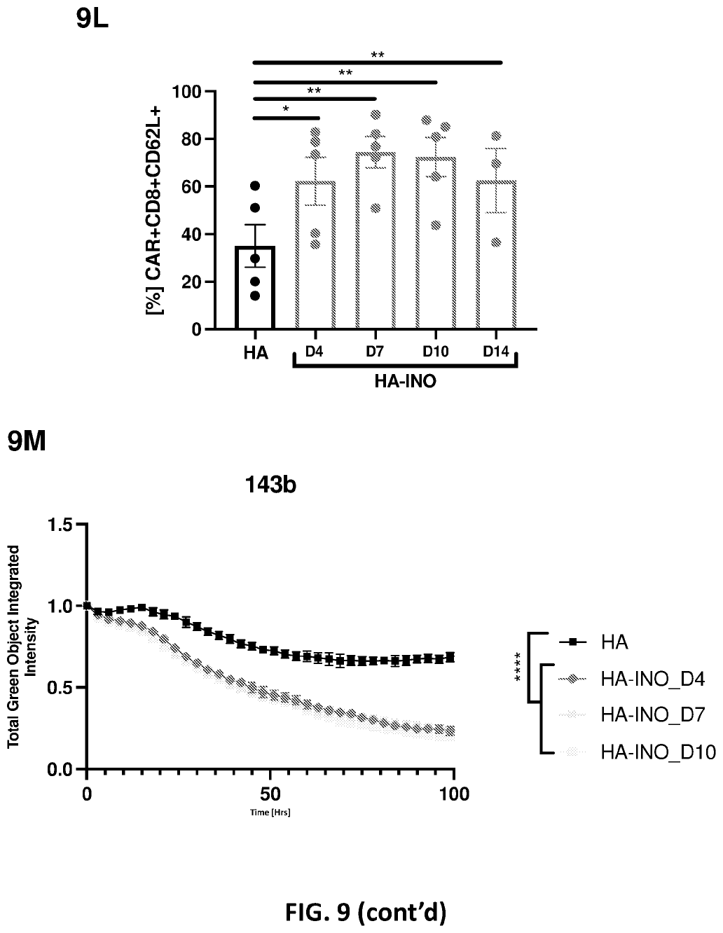

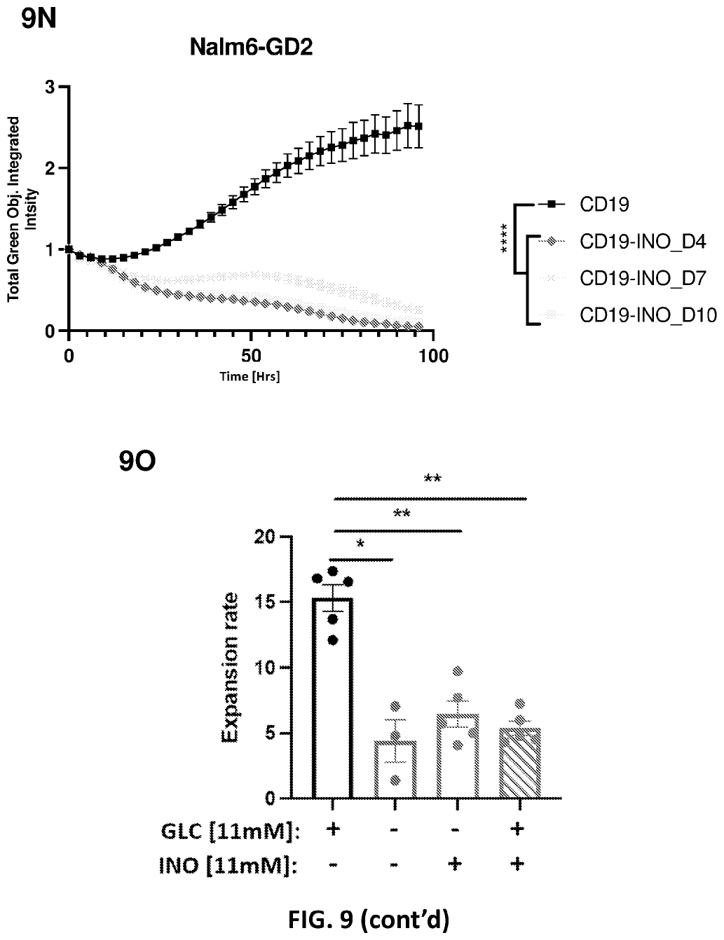

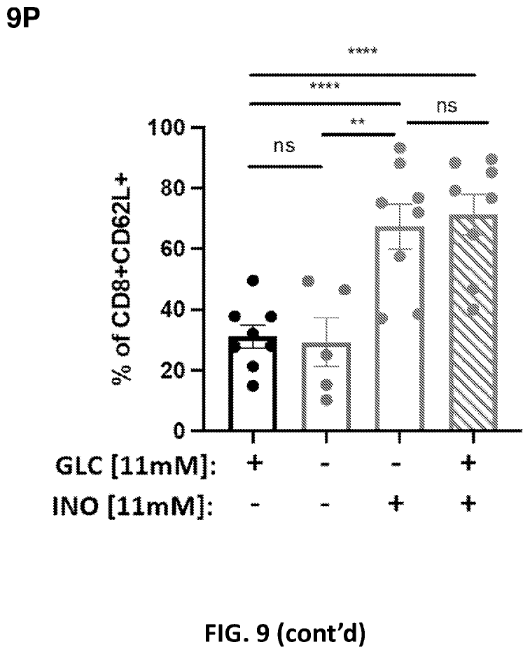

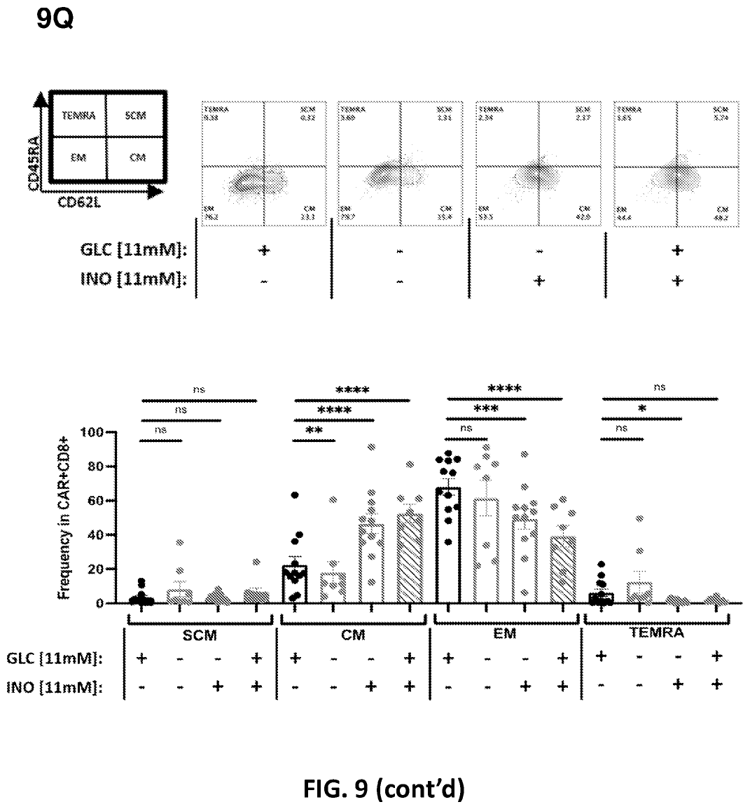

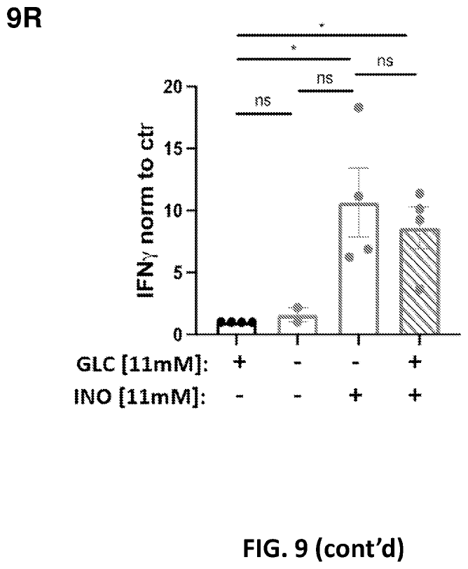

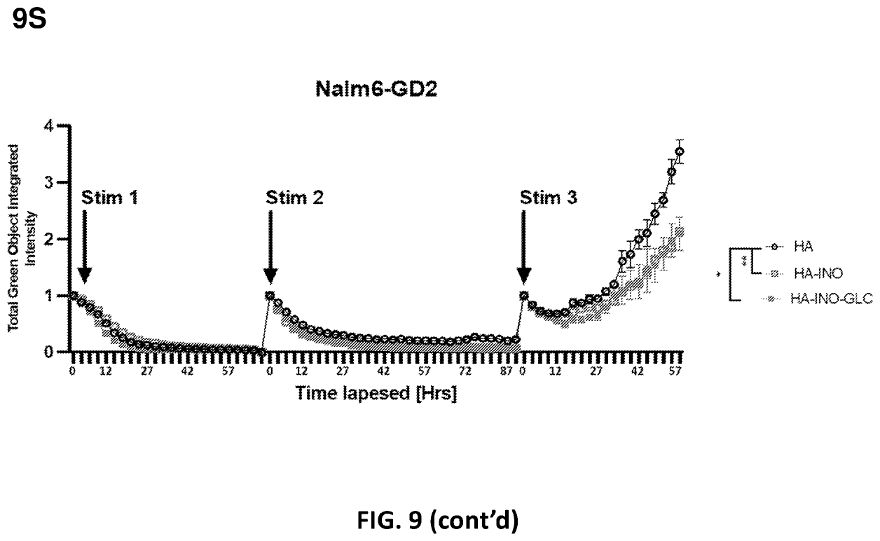

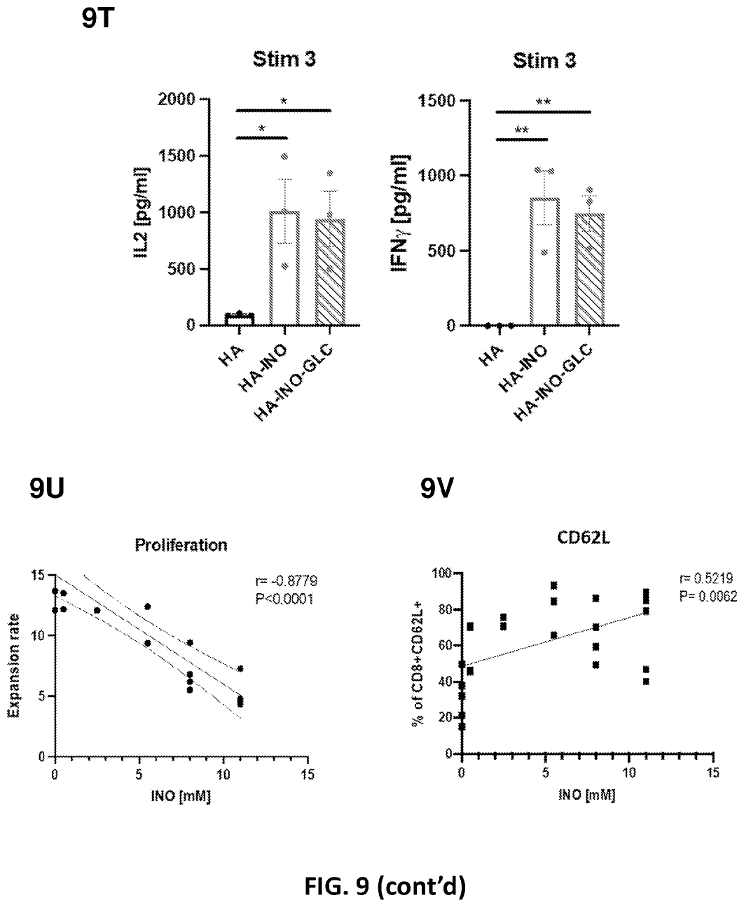

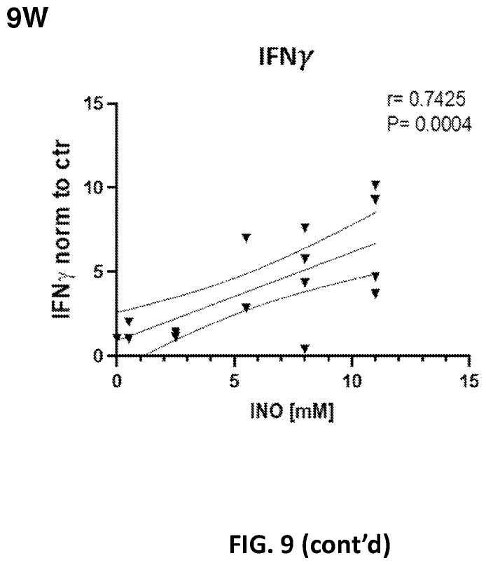

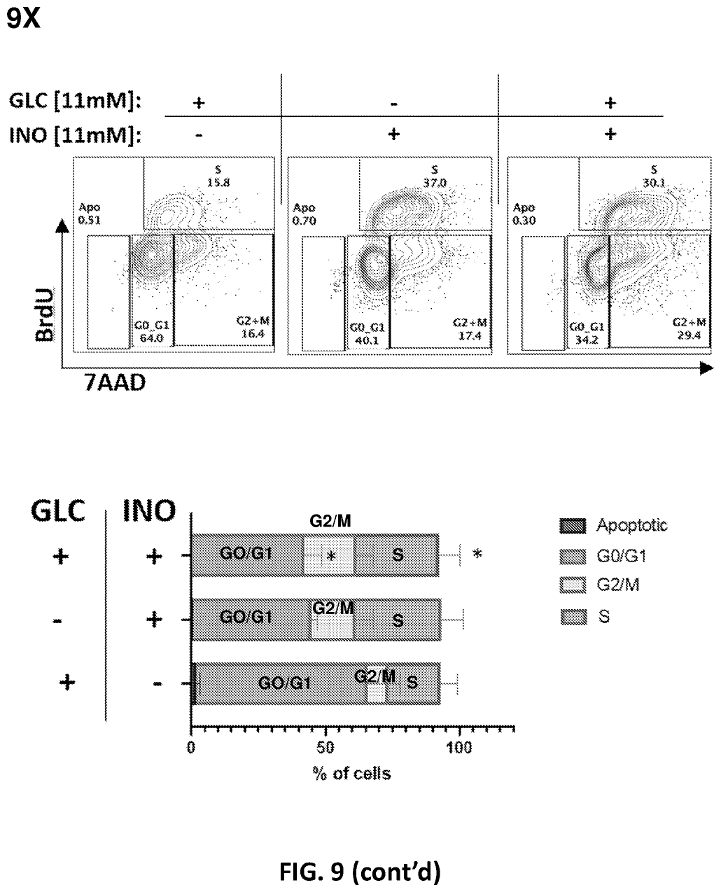

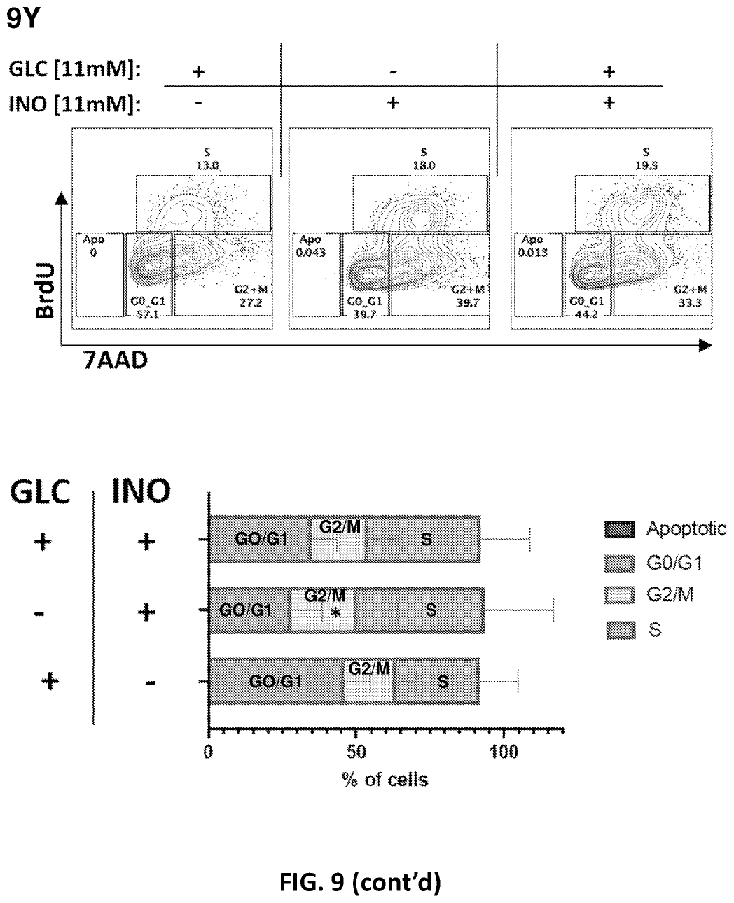

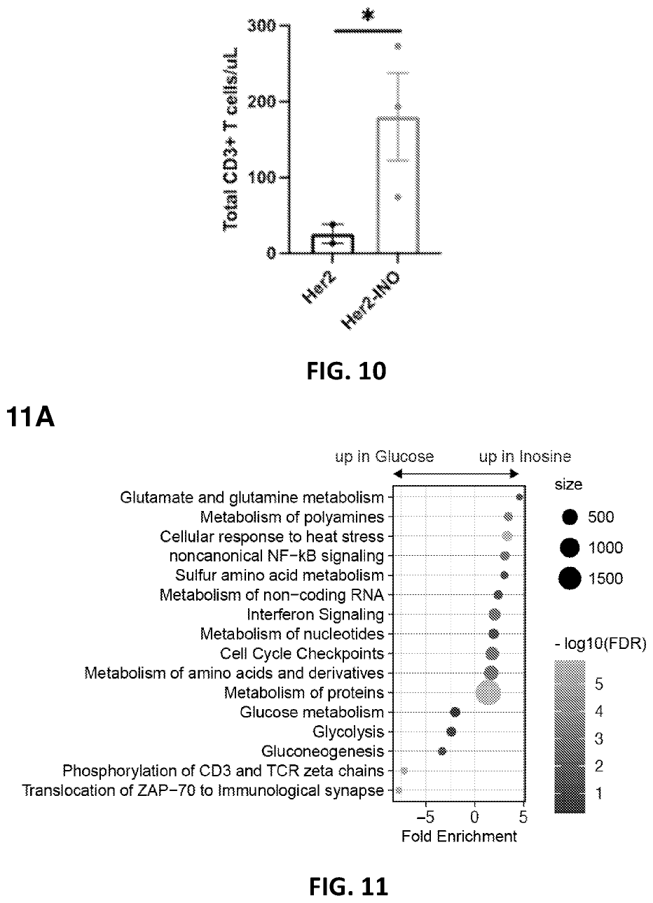

To test the hypothesis that INO could directly modulate stemness in chronically activated CAR T cells, cells were activated, transduced and cultured in standard RPMI containing 11 mM of glucose (unchanged from previous experiments) and, on day 4, cultures were split into standard RPMI or RPMI containing 11 mM of INO, without glucose (HA-INO-CAR) (FIG. 9A). Transcriptomic analysis on day 14 demonstrated that HA-INO-CAR T cells manifested differential expression of >3,000 gene transcripts compared to cells expanded in control media, with INO exposure responsible for 41.3% of variance observed in principal component analysis (PCA) (FIG. 9B). GSEA analysis demonstrated that HA-INO-CAR T cells upregulated transcriptional programs associated with stem cell-like memory (FIG. 9C) and downregulated genes involved in the purinergic pathway CD73 (NT5E), A2aR (ADORA2A) and FOXP3 (FIG. 9D). Flow cytometry following INO exposure demonstrated higher levels of CD62L (FIG. 9E) and increased protein expression of stemness markers (CD45RO CCR7, and CD127) in CD8+ and CD4+HA-INO-CAR T cells was confirmed by CyTOF analysis (FIG. 9F).

Functional studies following challenge with tumor cell lines expressing high (Nalm6-GD2) or low (143b) levels of GD2 (FIG. 9G) revealed increased cytotoxicity and cytokine production by both ADA-OE and HA-INO-CAR vs. control HA-CAR T cells (FIG. 9H-I), with HA-INO-CAR T cells showing highest potency. Also assessed were suppressive effects of HA-INO-CAR T cells and it was observed that CD19-CAR T cells activated in the presence of control HA-CAR T cells exhibited significantly decreased secretion of IL-2, whereas those activated in the presence of HA-INO-CAR T cells demonstrated increased IL-2 secretion compared to control (FIG. 9J).

Transcriptomic and epigenetic hallmarks of exhaustion occur within 10-12 days in the HA-CAR model. To determine if INO can induce a stem-like phenotype in already exhausted T cells, HA-CAR T cells were exposed to INO on day: 4, 7, 10 and 14 post-activation, analyzing phenotype and function (FIG. 9K). Observed was an increased frequency of CD8+CD62L+HA-CAR T cells, and increased cytotoxic function regardless of when INO was added, providing evidence that INO can enhance stemness even in cells already manifesting features of exhaustion (FIG. 9L, 9M). Similar enhanced cytotoxic function was observed in non-exhausted CD19-CAR T cells when challenged with Nalm6-GD2 tumor (FIG. 9N).

Sought next was to determine whether the effects observed with INO supplementation could be attributed to glucose deprivation, since transient glucose restriction has been demonstrated to augment T cell function. HA-CAR T cells were cultured from day 4 post-activation in control media containing glucose (11 mM), inosine only at 11 mM (INO), media containing both inosine and glucose at 11 mM or media without glucose or inosine. INO diminished expansion (FIG. 9O), enhanced CD62L expression (FIG. 9P) and the frequency of central memory cells (FIG. 9Q) and enhanced antigen induced IFNγ secretion (FIG. 9R) both in the presence or absence of glucose, demonstrating that the effects observed could not be attributed to glucose deprivation. Furthermore, while glucose deprivation induced a similar reduction in expansion compared to INO supplementation, glucose deprivation did not induce significant levels of CD62L, central memory cell expansion or enhanced antigen-specific activation.

Further tested was whether similar functional enhancement occurred with INO exposure in the presence or absence of glucose by serially challenging HA-INO-CAR cells grown in the presence or absence of glucose with Nalm6-GD2. Observed was enhanced tumor killing and cytokine secretion after multiple stimulations in the INO exposed cultures compared to controls, regardless of whether glucose was present or absent during the CAR-T cell manufacturing process (FIGS. 9S, 9T). Next, tested was the dose response effects of INO in regular RPMI containing glucose and observed a clear dose dependent effect on diminished proliferation, increased CD62L expression and increased antigen induced IFNγ production between 0-11 mM INO concentration without a plateau (FIG. 9U-9W). Decreased proliferation can be associated with induction of stemness or cells entering quiescence state. Analysis of the cell cycle revealed that INO-CAR T cells had a higher proportion of cells in S/G2+M phase as compared to control cells in the presence or absence of glucose (FIGS. 9X and 9Y), supporting the hypothesis that inosine promotes stemness retention in proliferating T cells rather than causing quiescence. Together these data demonstrate INO restrains proliferation of activated T cells, induces a more stem-like phenotype, endows enhanced functionality and eliminates suppressive activity. These effects are distinct from the effects of glucose deprivation, since they occur in the presence or absence of glucose and while diminished expansion is replicated by glucose deprivation, the enhanced stemness and functionality induced by INO exposure does not occur following glucose deprivation.

### Example 8-Identification of a Therapeutic Window for Inosine-Supplemented Manufacture of CAR T Cells

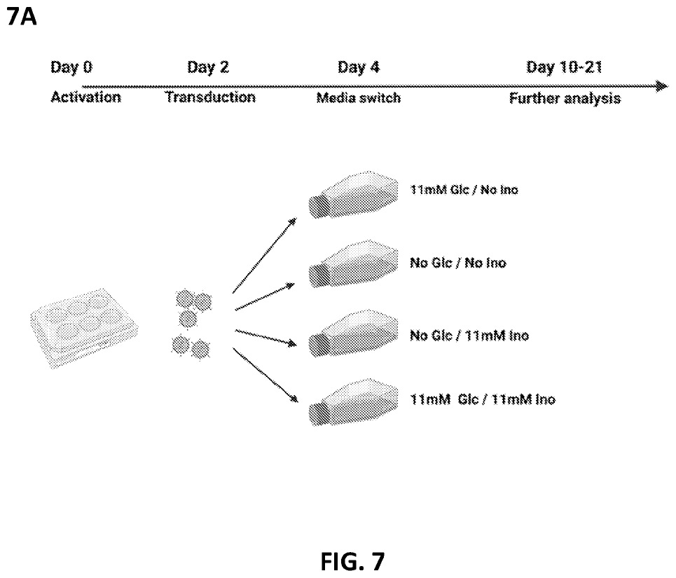

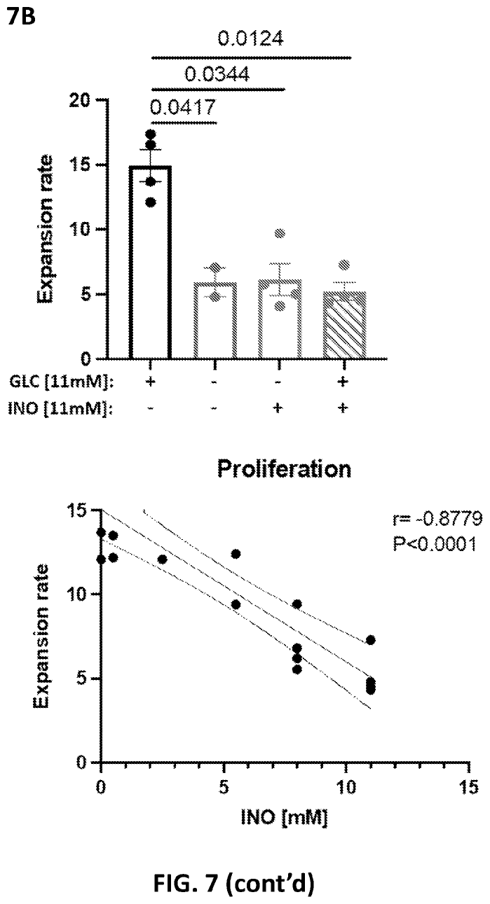

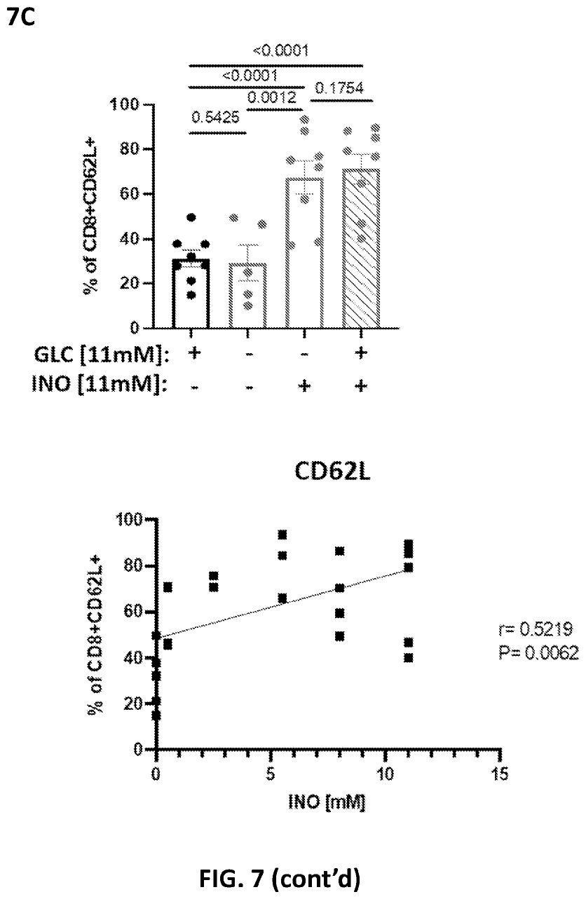

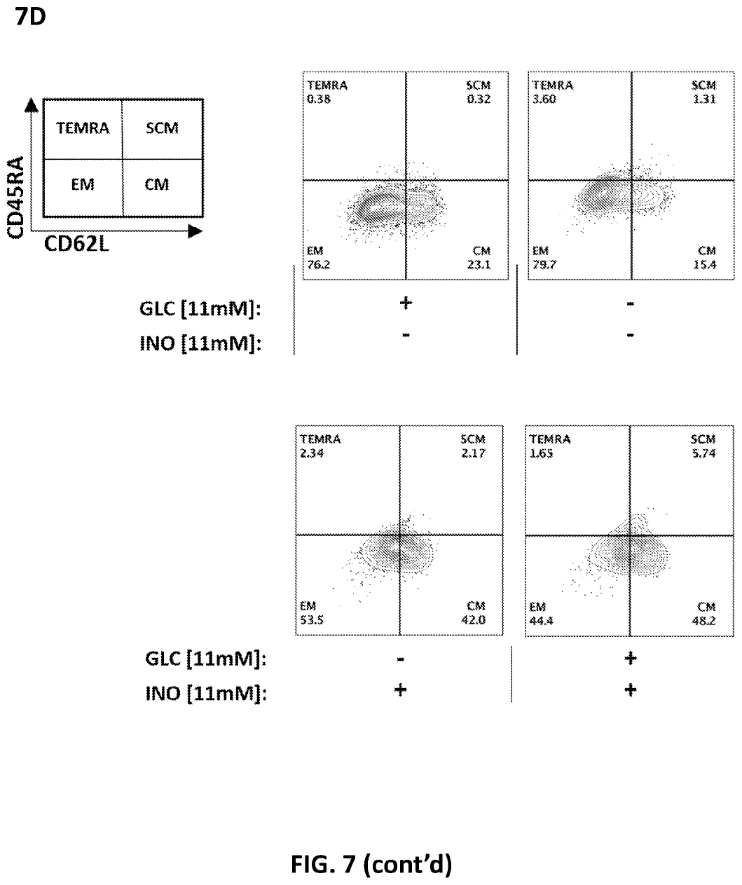

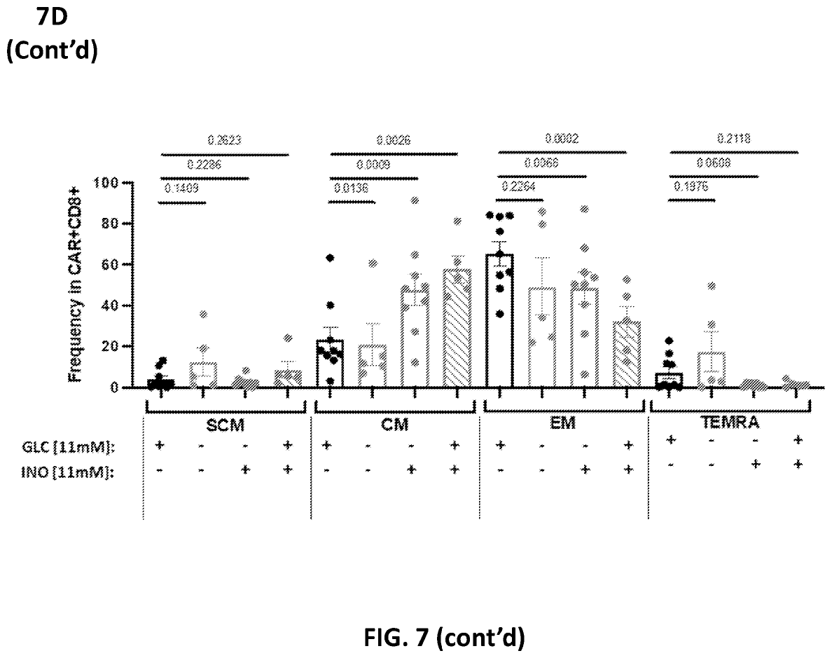

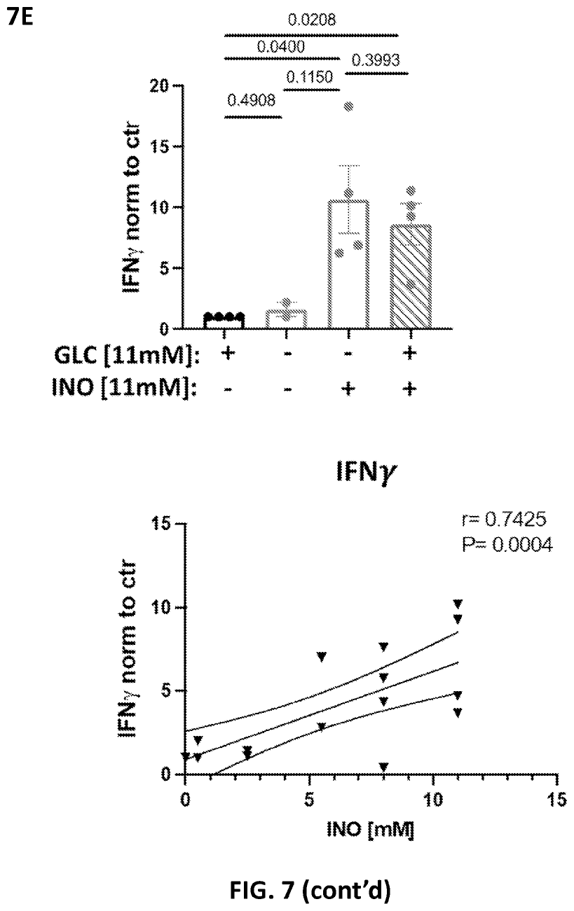

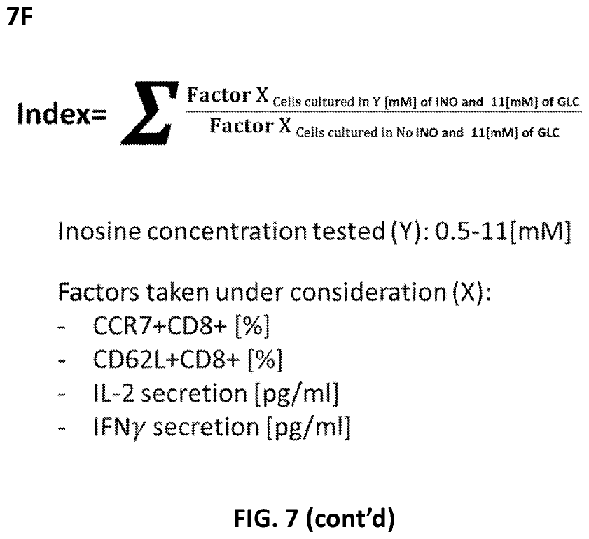

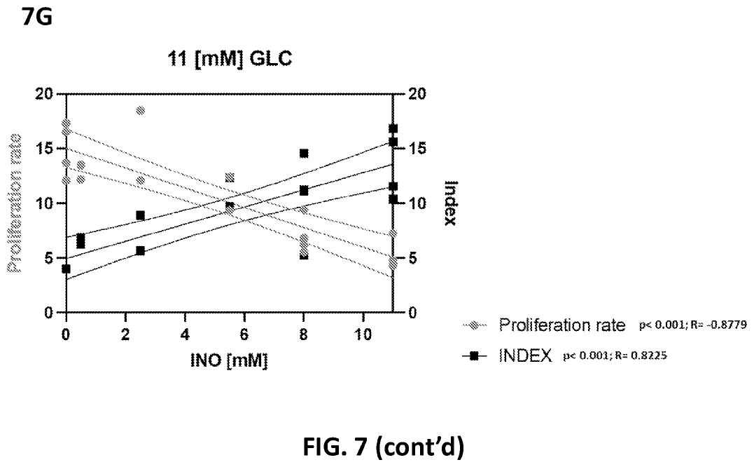

To assess whether the observed increase in stemness and functionality is attributable to inosine addition or glucose deprivation, HA-CAR T cells were cultured from day 4 post-activation in control media containing 11 mM glucose (CTR), no glucose and no inosine (starvation condition), 11 mM inosine only (INO), or media containing both inosine and glucose (FIG. 7A). Proliferation was assessed at day 10. INO significantly reduced expansion, whether or not glucose was present in dose-dependent manner (FIG. 7B). Phenotypic analysis revealed that inosine also significantly enhanced CD62L expression as well as the frequency of central memory CD8+HA-CAR T cells, independently of glucose (FIG. 7C-D). Inosine also significantly enhanced antigen induced IFNγ secretion in the presence or absence of glucose (FIG. 7E). Together these data demonstrate that inosine restrains proliferation of chronically activated T cells, induces a more stem-like phenotype, endows enhanced functionality in a dose-dependent manner, and that these effects are not replicated by glucose deprivation.

To establish a therapeutic window for inosine addition, an improvement index was calculated. The index represents a sum of fold change in frequency of CCR7+CD8+ and CD62L+CD8+ at the base line and fold change in IL-2 and IFNγ secretion after 24 hours stimulation with Nalm6-GD2 tumor line by HA-CAR T cells manufactured in 11 mM of glucose and various concentrations of inosine (0.5-11 mM) as compared to control (FIG. 7F). By overlapping the improvement index with the proliferation, it was concluded that the range of 2.75-11 mM of inosine concentration will significantly improve the phenotype and function of CAR T cells without a complete inhibition of cell expansion.

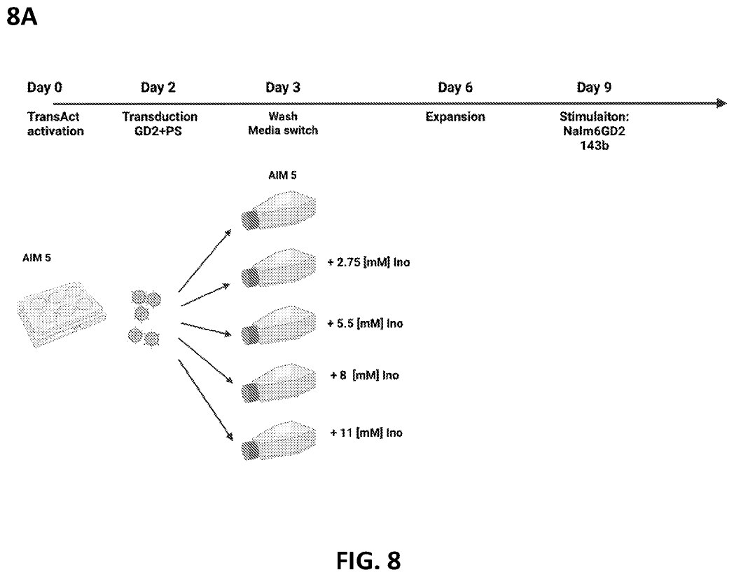

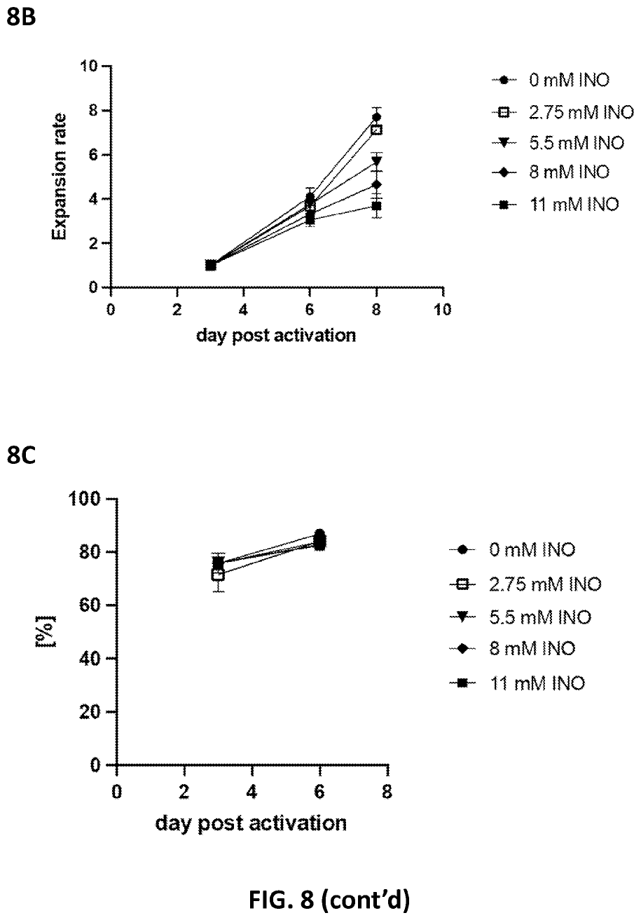

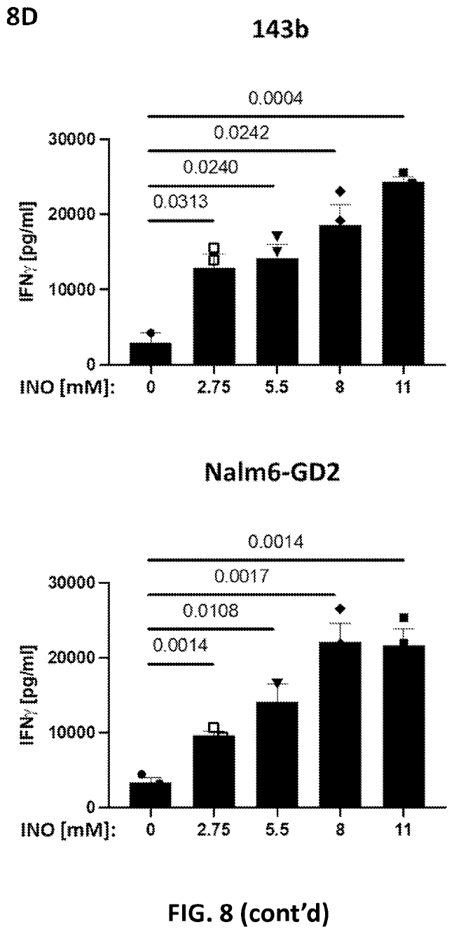

Next, to test this therapeutic window in the clinical context, GD2-CAR T cells were manufactured and determined to have anti-tumor activity in diffuse midline gliomas (DMGs) using GMP-grade AIM 5 media with increasing concentrations of INO (FIG. 8A). As expected, significantly decreased cell proliferation was observed (FIG. 8B) with no significant changes in CAR T cell viability (FIG. 8C). Inosine induced an increase in secretion of IFNγ when cells were challenged with 143b tumor line (low GD2 antigen density) or Nalm6-GD2 (high GD2 antigen density) (FIG. 8D). Together, these data demonstrate that CAR T cells exhibit enhanced functionality when manufactured in media containing 2.75-11 mM of inosine.

### Example 9-Inosine Modifies Metabolic Programming of Chronically Activated CAR T Cells

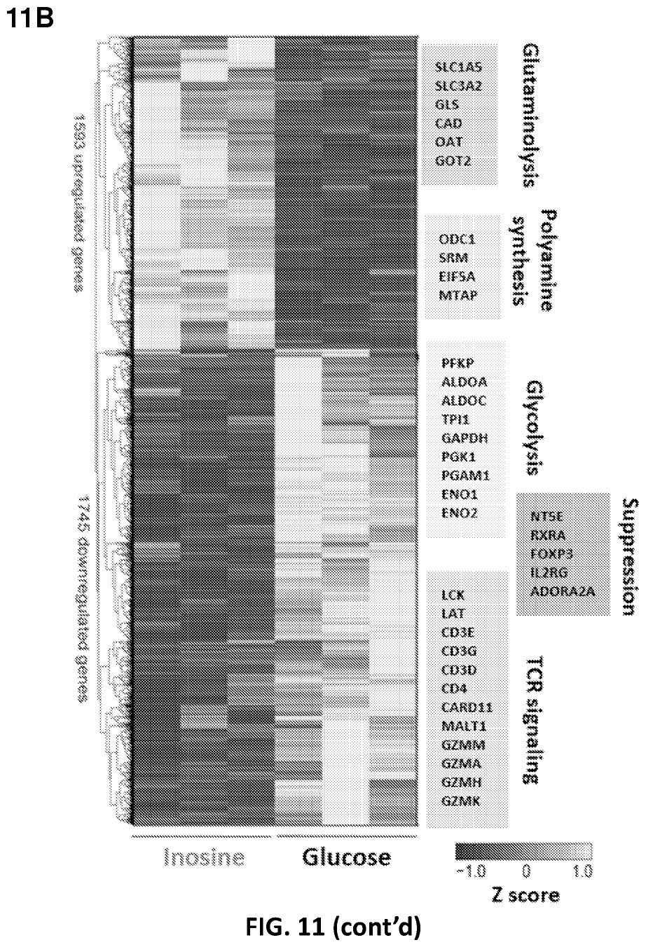

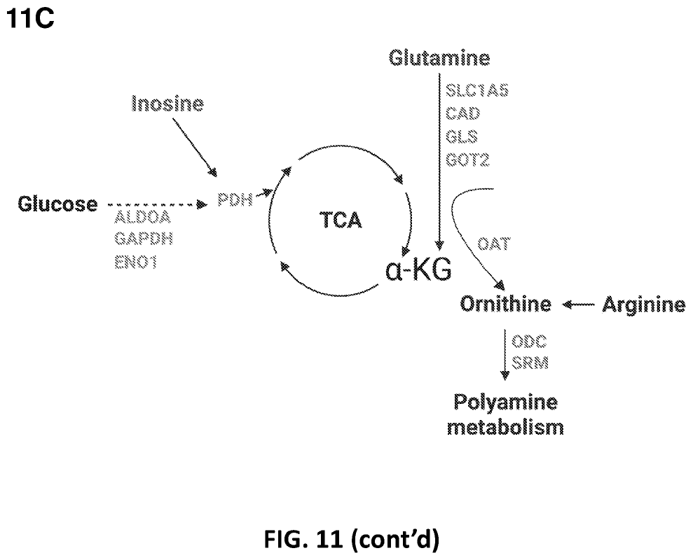

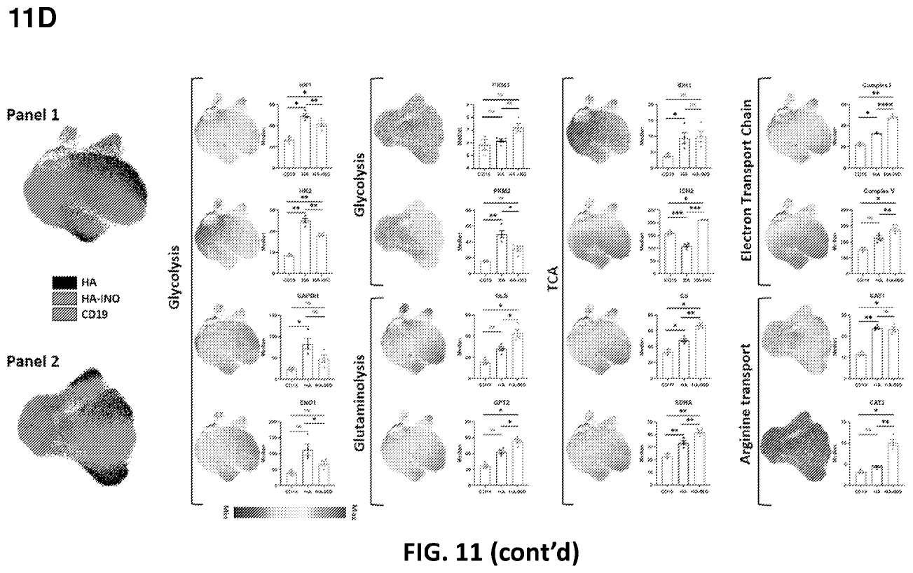

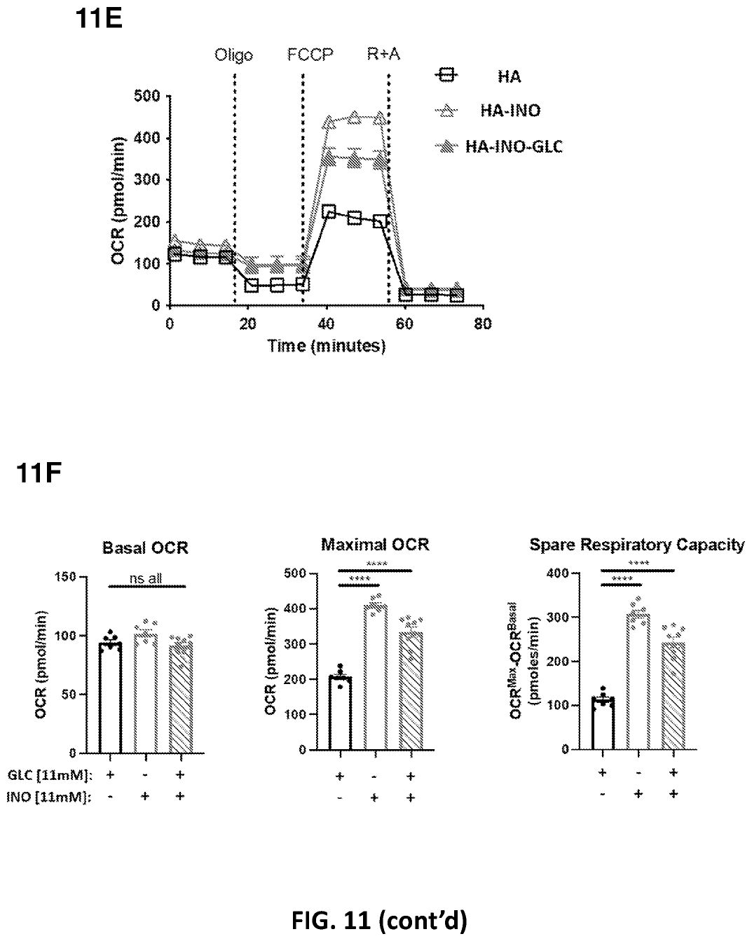

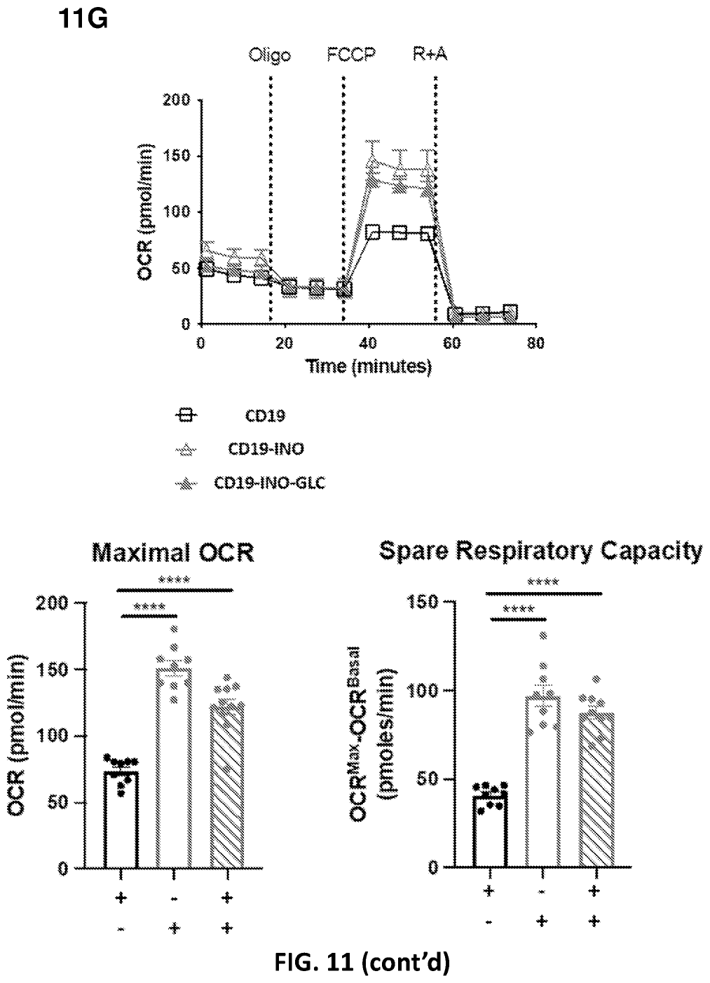

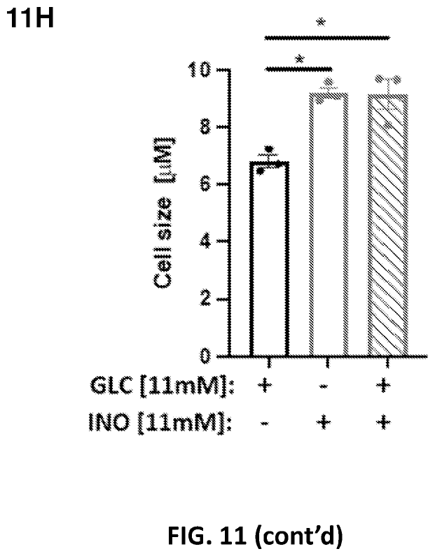

Inosine can serve as alternative source of carbon for T cells. Investigated here were the effects of INO on HA-CAR T cell metabolism. DAVID gene annotation enrichment analysis was first conducted using KEGG pathways and GO terms (FIG. 11A) and observed substantial evidence for metabolic reprogramming, with downregulation of genes associated with glycolysis and glucose driven metabolism (PFKP, ALDOA, GAPDH, PGK, ENO1), and increased expression of genes associated with glutaminolysis and polyamine synthesis (FIG. 11B-C). INO increased expression of ornithine decarboxylase (ODC), the proximal, rate limiting enzyme for putrescine synthesis and spermidine synthase, which generates spermidine and elF5A, important mitochondria activity regulators. These metabolic effects were associated with diminished expression of genes associated with TCR signaling consistent with decreased effector differentiation.

To assess whether protein expression also demonstrated evidence for metabolic reprogramming, mass cytometry was used to perform single cell metabolic regulome profiling using two panels of antibodies targeting metabolic enzymes and nutrient transporters (FIG. 11D). Compared to HA-CAR T cells, the proteome of HA-INO-CAR T cells demonstrated reduced levels of enzymes associated with glycolysis, including reduced hexokinases (HK1/2), glyceraldehyde 3-phosphate dehydrogenase (GAPDH), enolase 1 (ENO1) and PKM2. Importantly however, glycolysis was not completely inhibited, since glycolytic enzymes were present at similar levels in HA-INO-CAR T cells and control CD19-CAR T cells, consistent with previous evidence demonstrating that INO is capable of providing a carbon source for glycolysis. INO cultured cells also expressed higher levels of enzymes associated with glutaminolysis (GLS, GPT2), and TCA activity (IDH1, IDH2, CS, SDHA), consistent with INO induced glutamine catabolismdriving TCA through anapleurosis (FIG. 11D). Proteins of complex I and complex V of the electron transport chain were also elevated, consistent with higher oxidative phosphorylation. Expression of CAT2, a high efficiency transporter of the polyamine precursor-arginine also increased following INO culture, providing further evidence in support of increased polyamine synthesis (FIG. 11D).

To measure mitochondrial fitness in CAR T cells cultured in the presence or absence of INO, Seahorse analysis was performed. Basal oxygen consumption rate (Basal OCR) did not differ between control and INO cultured cells, however HA-INO-CAR T cells exhibited increased maximal OCR and spare respiratory capacity (SRC), both hallmarks of memory-like cells (FIG. 11E-11F). Similarly, INO induced a memory-like metabolic profile in non-tonic signaling CD19-CAR T cells (FIG. 11G). Increase in mitochondrial activity correlated with an increased size of the cells in INO condition (FIG. 11H). Together, phenotypic, transcriptomic and proteomic profiling demonstrate that INO induced stemness programming and augmented functionality is associated with profound metabolic reprogramming, characterized by diminished glycolysis, increased glutaminolysis and polyamine synthesis, increased TCA and mitochondria activity.

### Example 10-Inosine Modulates the Epigenetic Landscape Toward Stemness

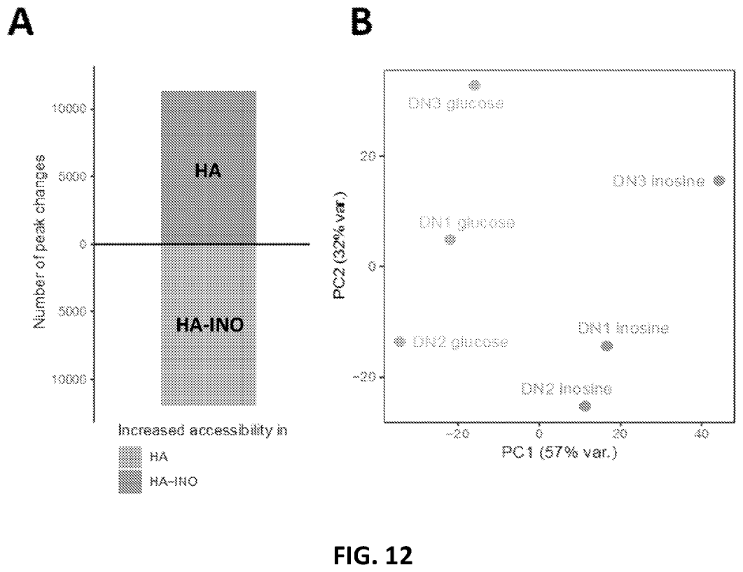

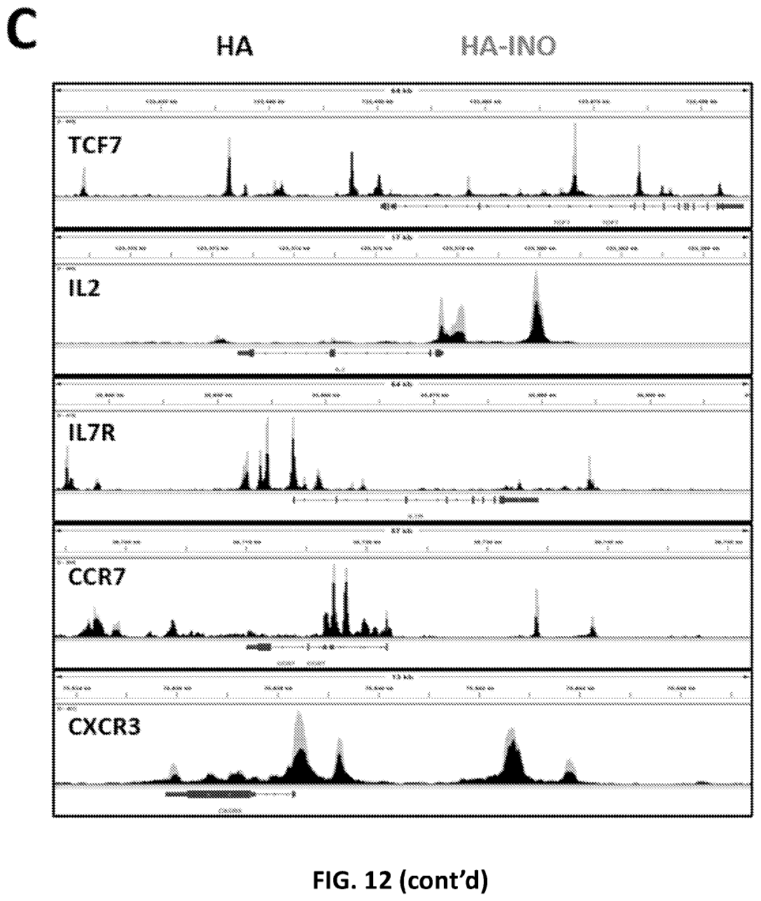

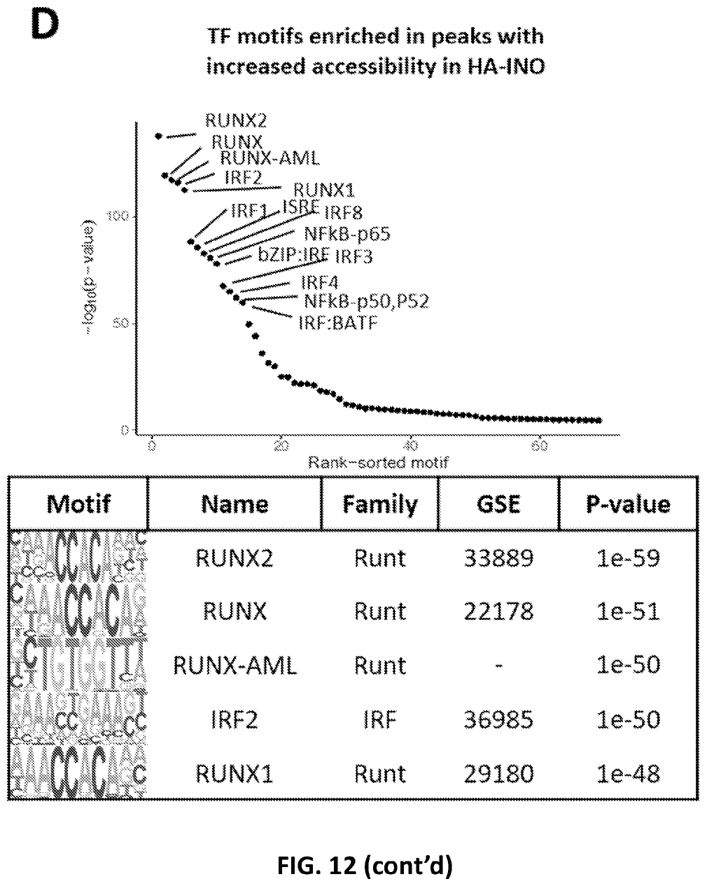

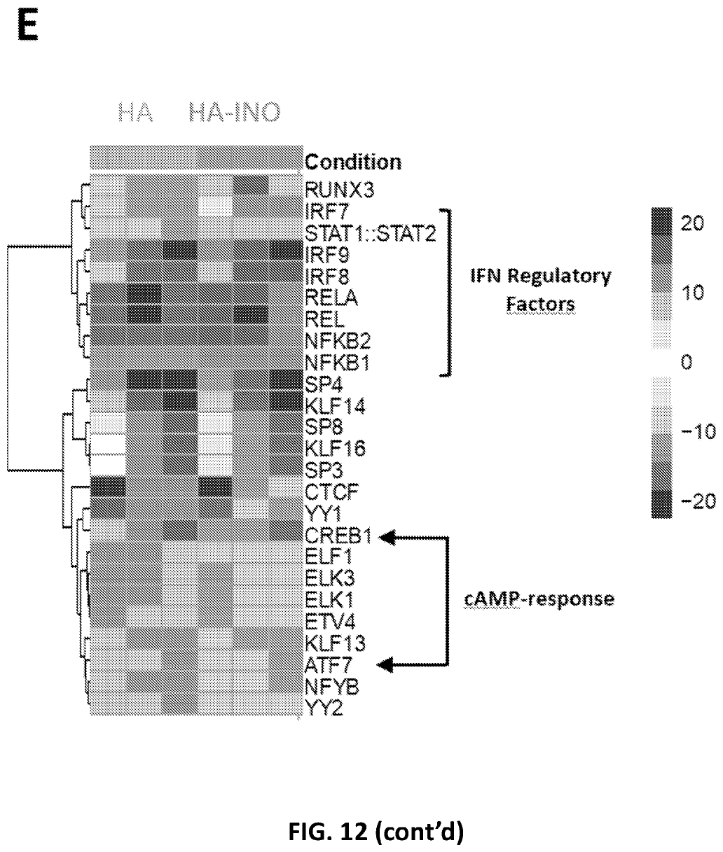

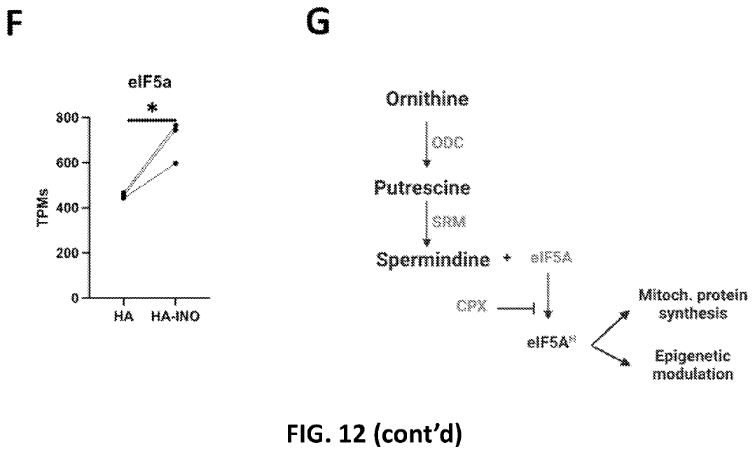

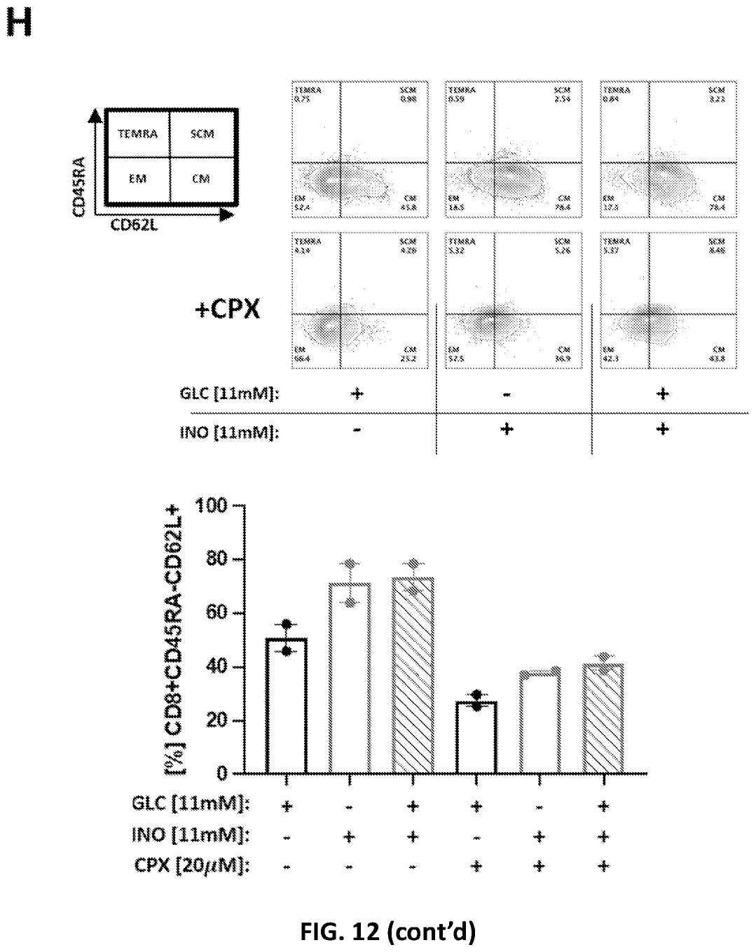

T cell exhaustion is associated with widespread epigenetic changes and metabolic pathways are increasingly implicated in modulating the epigenome of T cells. To assess whether INO induced functional and metabolic reprogramming was associated with epigenetic reprogramming of exhausted T cells, ATACseq was used to compare the chromatin landscape of CD8⁺ HA- and HA-INO-CAR T cells. INO induced widespread changes in chromatin accessibility spanning more than 10,000 genomic regions (PC1 variance 57%) (FIG. 12A-B). Enhanced chromatin accessibility in HA-INO-CAR T cells was evident in regulatory regions associated with IL-2 and several stemness genes (TCF7, IL-7R, CCR7 and CXCR3) (FIG. 12C). Using ChromVAR and HOMER transcription factor binding motif enrichment analysis, it was discovered that INO also increased accessibility of RUNT (RUNX1/2), IRF (IRF1/2/3/4/8, ISRE) and RHD (NFKB1/2, REL (A)) family TF binding motifs, all which are associated with memory differentiation (FIG. 12D). In contrast, accessibility of cAMP-response element binding proteins CREB1 and ATF7 were decreased in INO-HA compared to HA-CAR T cells, raising the prospect that INO exposed cells are less susceptible to Ado-mediated suppression (FIG. 12E). Previous work has implicated polyamines in epigenetic regulation of Th1 differentiation and regulation of IFNg production through hypusination of translation elongation factor elF5A. Observed was significantly increased expression of translation elongation factor elF5A in HA-INO-CAR T cells suggesting that a similar pathway may be involved in INO induced epigenetic programming (FIG. 12F). To test whether hypusination of elF5A was required for the effects of INO in our model, ciclopirox (CPX), a small molecule that inhibits the deoxyhypusine hydroxylase (DOHH) enzyme required for elF5A hypusination was used (FIG. 12G). Treatment of inosine-cultured HA-CAR T cells with CPX resulted in striking decrease in frequency of central memory-like cells (FIG. 12H). Together, the data demonstrate that INO induces metabolic and epigenetic programming in T cells that drive enhanced functionality and induces features of stemness and implicate polyamine metabolism and hypusination of elF5A in INO's mechanism of action.

### Example 11-Manufacturing of Clinical Grade CAR T Cells Using Inosine-Containing Media

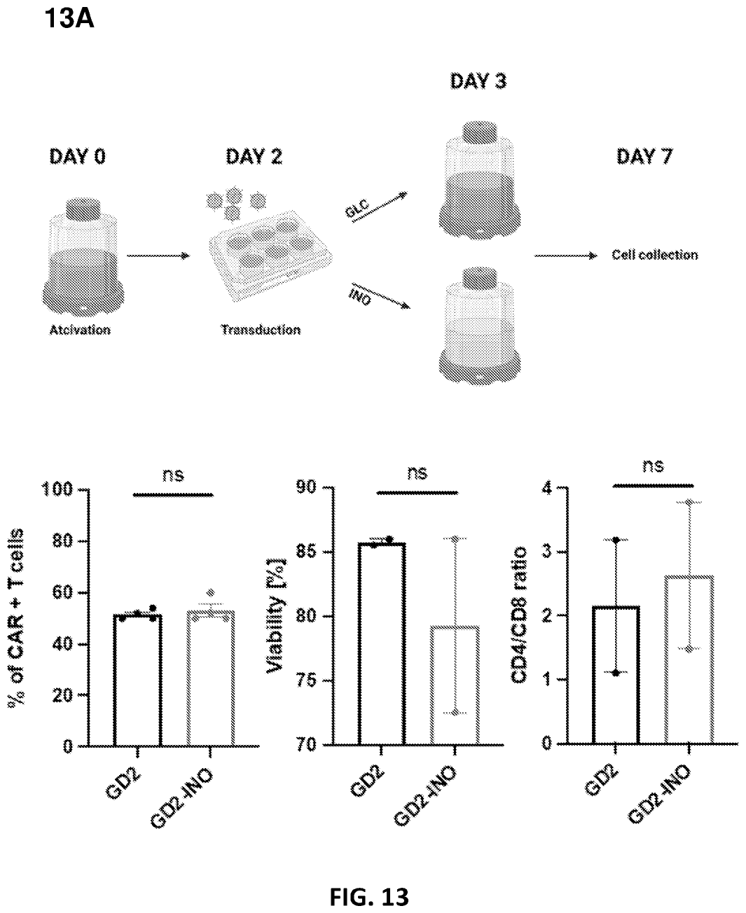

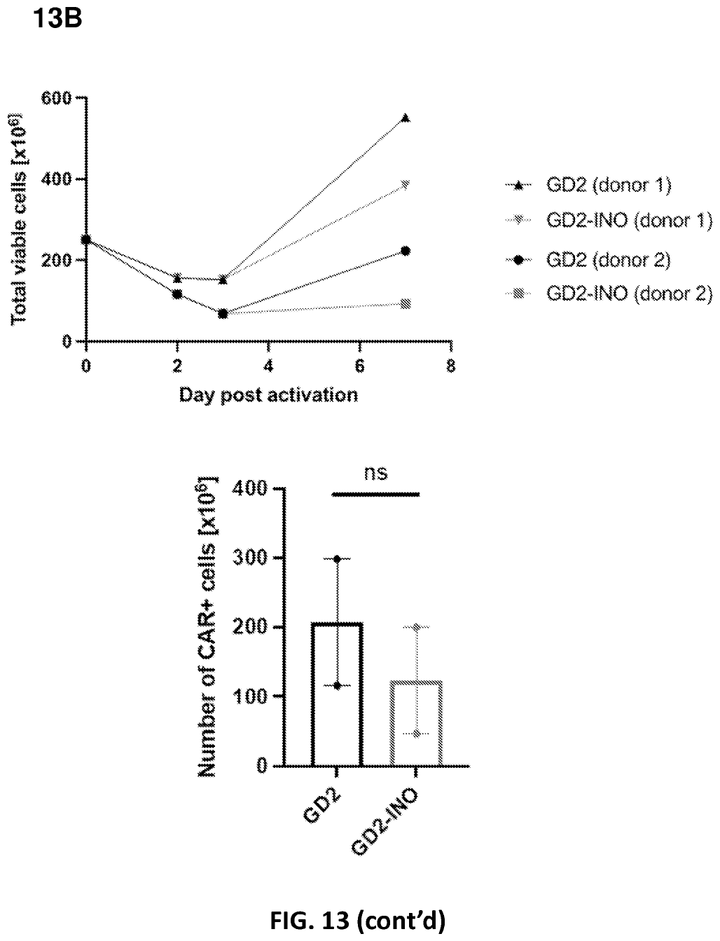

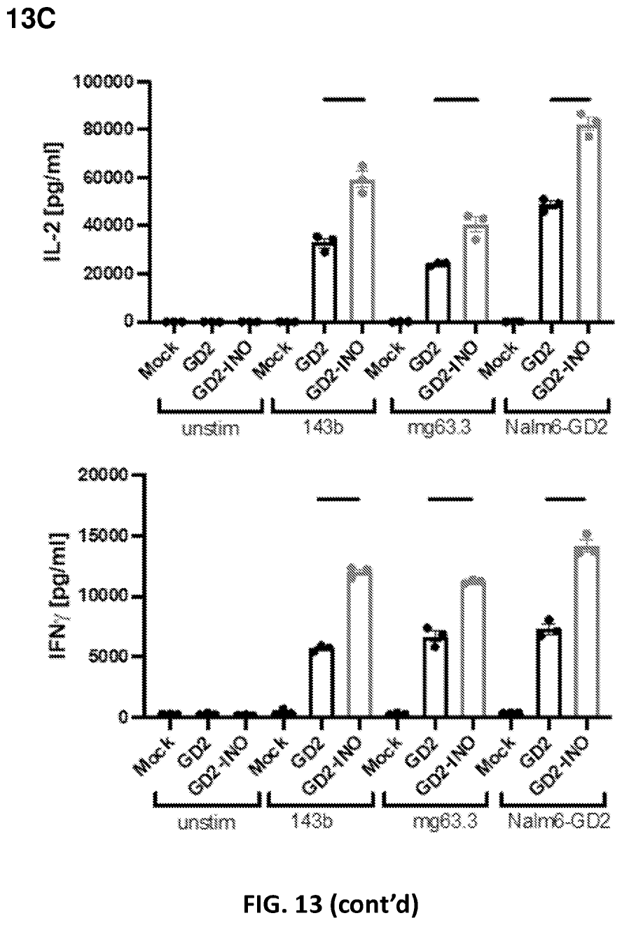

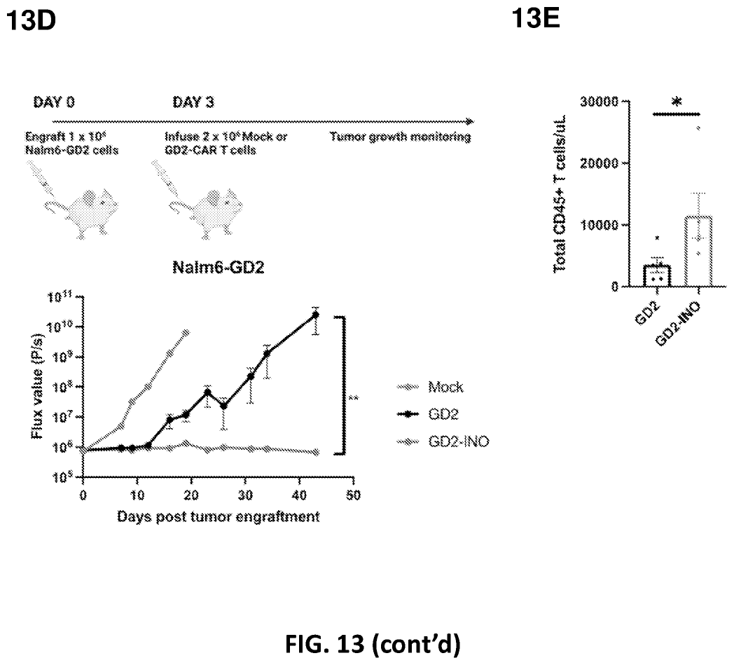

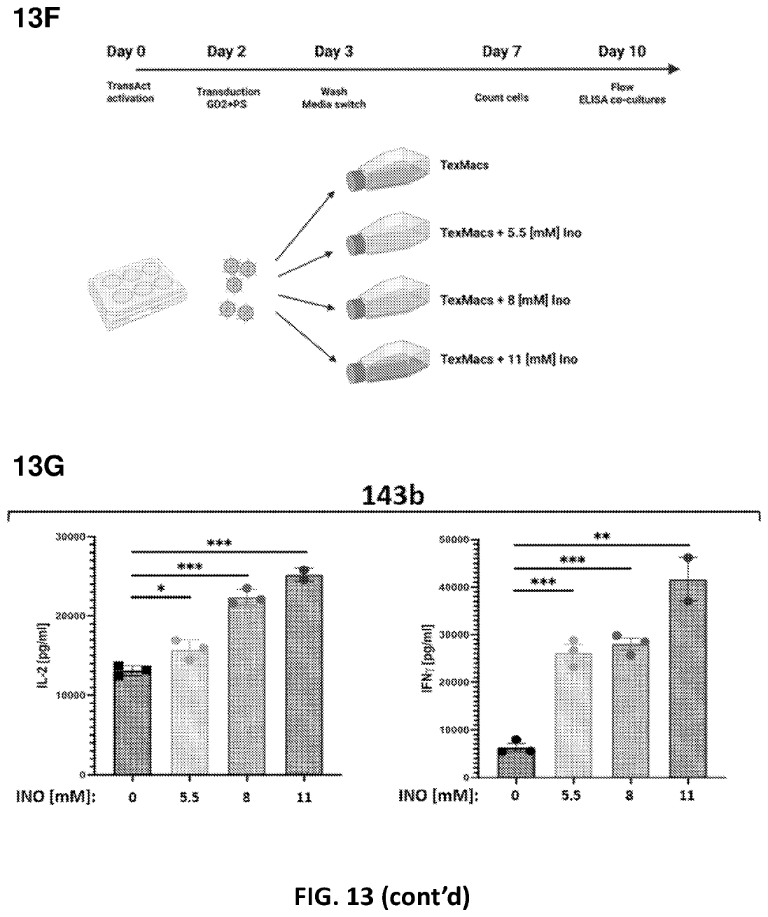

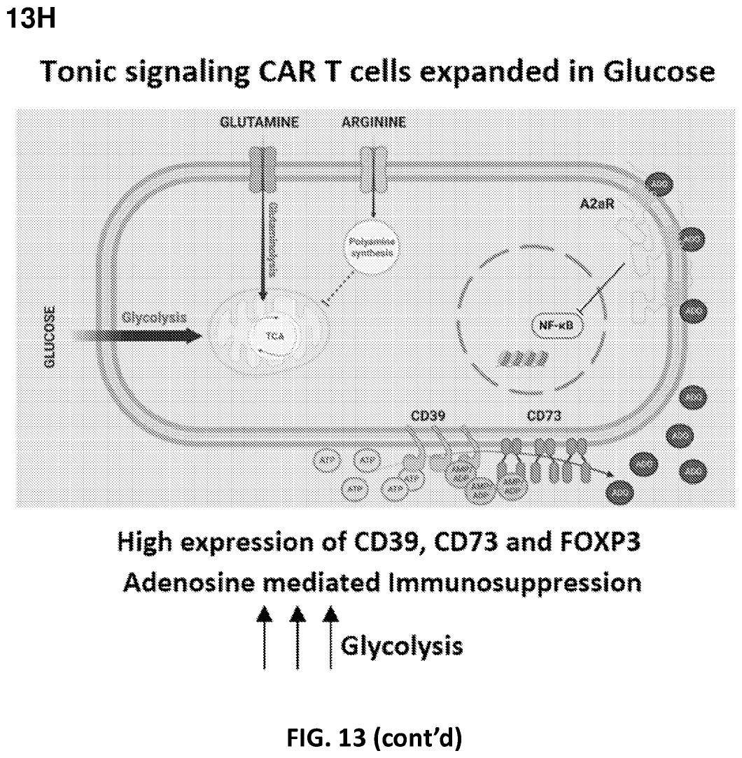

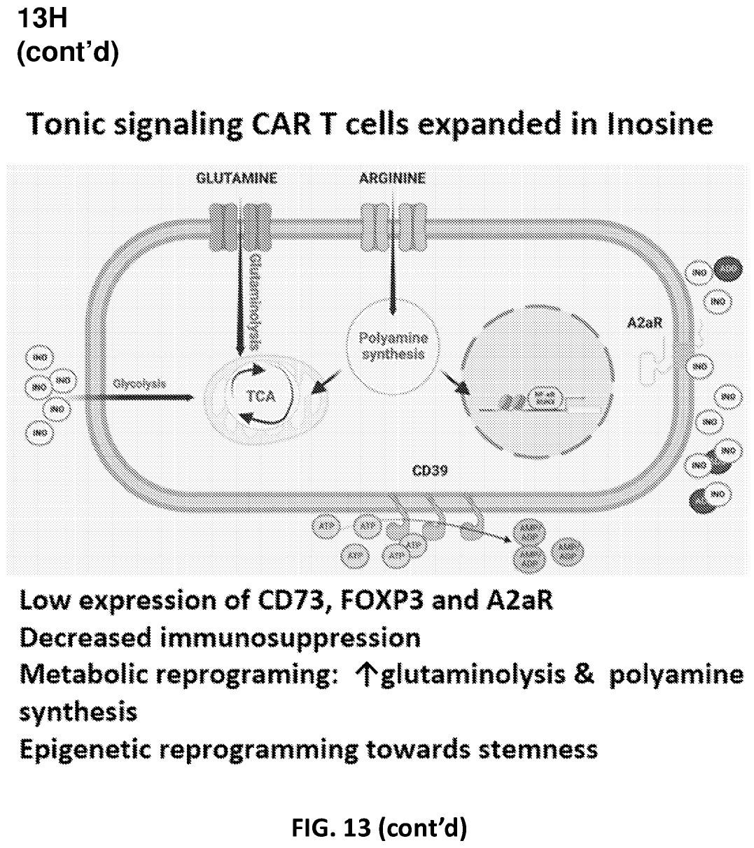

Tested in this example was whether it is feasible to manufacture GD2-CAR T cells, which have demonstrated anti-tumor activity in diffuse midline gliomas (DMGs) using INO in a GMP compliant process at clinical scale. A total of 2.5×10⁸ enriched CD4+ and CD8+ T cells from a healthy donor were activated with anti-CD3/CD28-coated beads and transduced using clinical grade GD2-CAR vector at an MOI of 10 (FIG. 13A). On day 3, T cells were maintained in standard RPMI media containing glucose or switched to inosine-containing RPMI without glucose in a semi-closed G-Rex culture platform. On day 7, there was no difference in transduction efficiency, CD4/CD8 ratio or viability between control and INO CAR+ T cells (FIG. 13A). The total cell yield in INO cultures was reduced compared to control cultures as predicted (238.5×10^{6+145.8} vs. 387.7×10⁶⁺¹⁶⁵ s.e.m., respectively), however, the yields were sufficient to formulate clinical doses of 1×10⁶ CAR+ T cells/kg body weight (+/−20%) (FIG. 13B). Consistent with the results from the small-scale experiments, observed was a higher frequency of naive and central memory GD2-CAR T cells in INO cultures. When stimulated with 143b (low GD2 expression), mg63.3 or Nalm6-GD2 (high GD2 expression) tumor cells, GD2-INO-CAR T cells secreted significantly more IL-2 and IFNg (FIG. 13C), mediated significantly greater antitumor effects in vivo (FIG. 13D), and exhibited greater persistence when compared to control GD2-CAR T cells (FIG. 13E).

Finally, to test compatibility of adding INO to GMP-grade culture media that contains glucose, GD2-CAR T cells were manufactured in TexMACS™ with increasing concentrations of INO (FIG. 13F). Observed was significantly decreased cell proliferation but no changes in CAR T cell viability. INO increased CD62L expression compared to control cells and a dose-dependent increase in secretion of IL-2 and IFNg was observed when cells were challenged with 143b cells (FIG. 13G). While INO concentrations between 5-11 mM mediated similar anti-proliferative effects and CD62L induction, a trend toward greater antigen induced cytokine production with INO concentrations of 11 mM was observed (FIG. 13G). Together, these data demonstrate that CAR T cells can be manufactured in INO containing media using a GMP adaptable process to reach clinically relevant doses that meet standard release criteria and that such cells demonstrate enhanced functionality compared to those manufactured using standard glucose-based media.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of manufacturing therapeutic cells, the method comprising:
expanding cells in the presence of inosine; and
harvesting the cells after expansion.

2. The method according to claim 1, wherein the cells are expanded in a culture medium comprising from 2 mM to 20 mM inosine.

3. The method according to claim 2, wherein the cells are expanded in a culture medium comprising from 2 mM to 12 mM inosine.

4. The method according to claim 3, wherein the cells are expanded in a culture medium comprising from 2.75 mM to 11 mM inosine.

5. The method according to claim 2, wherein the cells are expanded in a culture medium comprising from 7 mM to 15 mM inosine.

6. The method according to any one of claims 1 to 5, wherein the cells are expanded in a culture medium supplemented with inosine at from 0 to 7 days post-activation.

7. The method according to claim 6, wherein the cells are expanded in a culture medium supplemented with inosine at from 2 to 5 days post-activation.

8. The method according to any one of claims 1 to 7, wherein the cells are expanded in the presence of inosine for from 12 hours to 14 days.

9. The method according to any one of claims 1 to 8, wherein the cells are expanded in the presence of inosine in a culture medium that comprises less than 15 mM glucose.

10. The method according to claim 9, wherein the cells are expanded in the presence of inosine in a culture medium that comprises no glucose.

11. The method according to any one of claims 1 to 10, wherein prior to, during, or subsequent to expanding the cells in the presence of inosine, the cells are genetically modified.

12. The method according to claim 11, wherein the cells are genetically modified to express an engineered receptor.

13. The method according to claim 12, wherein the engineered receptor is a chimeric antigen receptor (CAR), an engineered T cell receptor (TCR), a chimeric cytokine receptor (CCR), a chimeric chemokine receptor, a synthetic notch receptor (synNotch), a Modular Extracellular Sensor Architecture (MESA) receptor, a Tango receptor, a ChaCha receptor, or a generalized extracellular molecule sensor (GEMS) receptor.

14. The method according to claim 13, wherein the engineered receptor is a CAR.

15. The method according to any one of claims 12 to 14, wherein the engineered receptor comprises an extracellular binding domain that binds to a tumor antigen.

16. The method according to any one of claims 1 to 11, wherein the cells are not genetically modified to express an engineered receptor.

17. The method according to any one of claims 1 to 16, wherein the cells are human cells.

18. The method according to any one of claims 1 to 17, wherein the cells are immune cells.

19. The method according to claim 18, wherein the immune cells are T cells.

20. The method according to claim 19, wherein the T cells are CD8+ T cells.

21. The method according to claim 19, wherein the T cells are CD4+ T cells.

22. The method according to claim 19, wherein the T cells are regulatory T cells (Tregs).

23. The method according to claim 18, wherein the immune cells are natural killer (NK) cells.

24. The method according to any one of claims 1 to 23, further comprising administering an effective amount of the manufactured cells to a subject in need thereof.

25. A population of cells manufactured according to the method of any one of claim 1 to 24.

26. The population of cells of claim 25 formulated for administration to a subject in need thereof.

27. A method comprising administering an effective amount of the population of cells of claim 26 to a subject in need thereof.

28. The method according to claim 27, wherein the subject in need thereof has cancer.

29. The method according to claim 28, wherein the cells are modified to express an engineered receptor comprising an extracellular binding domain that binds to a tumor antigen on the surface of cells of the cancer.

30. The method according to claim 29, wherein the engineered receptor is a chimeric antigen receptor (CAR), an engineered T cell receptor (TCR), a chimeric cytokine receptor (CCR), a chimeric chemokine receptor, a synthetic notch receptor (synNotch), a Modular Extracellular Sensor Architecture (MESA) receptor, a Tango receptor, a ChaCha receptor, or a generalized extracellular molecule sensor (GEMS) receptor.

31. The method according to claim 30, wherein the engineered receptor is a chimeric antigen receptor (CAR).

32. A kit, comprising:
inosine; and
instructions for expanding cells in a culture medium comprising the inosine.

33. The kit of claim 32, wherein the inosine is present in a cell culture medium.
